(19) United States
(12) Patent Application Publication
(10) Pub. No.: US 2010/0311734 A1
(43) Pub. Date: Dec. 9, 2010

(54) Spiro Compounds Useful as Antagonists of the H1 Receptor

(75) Inventors: Maurizio Botta, Siena (IT); Emiliano Castiglioni, Verona (IT); Romano Di Fabio, Verona (IT); Raffaella Spinosa, Vasto (IT); Andrea Togninelli, Rome (IT)

Correspondence Address: GlaxoSmithKline;GLOBAL PATENTS -US, UW2220, P. O. BOX 1539, KING OF PRUSSIA, PA, 19406-0939, US

(73) Assignee: GLAXO GROUP LIMITED

(21) Appl. No.: 12/670,704

(22) PCT Filed: Jul. 24, 2008
(86) PCT No.: PCT/EP08/59683

§ 371 Date: May 25, 2010

(30) Foreign Application Priority Data
Jul. 27, 2007 (GB) 0714713.5

(51) Int. Cl. A61K 31/5386; C07D 295/033; C07D 207/04; C07D 205/04; C07D 211/06; C07D 221/20; C07D 498/10; C07D 491/107; A61K 31/136; A61K 31/5375; A61K 31/40; A61K 31/495; A61K 31/54; A61K 31/451; A61K 31/438; A61K 31/403; A61P 25/00

(52) U.S. Cl. 514/227.5; 564/426; 544/70; 548/407; 544/230; 544/6; 548/950; 546/15; 546/17; 544/71; 548/409; 514/656; 514/231.2; 514/409; 514/255.03; 514/278; 514/230.8

(57) ABSTRACT

The invention provides a compound of formula (I) or a pharmaceutically acceptable salt thereof, for treating diseases and conditions of the central nervous system (CNS), in particular sleep disorders.

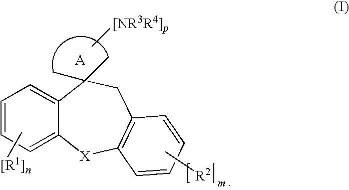

This invention relates to novel spiro derivatives. The invention also relates to the use of the derivatives in treating diseases and conditions of the central nervous system (CNS), in particular sleep disorders. In addition, the invention relates to compositions containing the derivatives and processes for their preparation.

Common symptoms for those suffering with a sleep disorder include abnormal sleep behaviour and difficulties in one or more of falling asleep, remaining asleep, sleeping for adequate lengths of time and achievement of restorative sleep.

Available treatments for sleep disorders include the use of prescription hypnotics, e.g., benzodiazepines. However, these may be habit-forming, lose their effectiveness after extended use, and metabolise more slowly for certain designated groups, resulting in persisting medicative effects.

Other treatments include over-the-counter antihistamines, e.g., diphenhydramine or dimenhydrinate. These are not designed to be strictly sedative in their activity and as such, this method of treatment has been associated with a number of adverse side effects, e.g., persistence of the sedating medication after the prescribed time of treatment, or the so-called “hangover effect”. Many of these side effects result from nonspecific activity in both the periphery as well as the CNS during this period of extended medication.

Therefore, a need exists for the development of new compounds useful for the improved treatment of sleep disorders.

It has been suggested that brain histamine is involved in the regulation of the sleep-wake cycle, arousal, cognition and memory mainly through H₁ receptors, producing a reduction of the sleep latency in both preclinical (Shigemoto et al., (2004), Eur J. Pharmacol., 494(2-3):161-5) and clinical studies (Simons et al., (1996), Clin Exp Allergy, 26(9):1092-7).

In parallel, selective blockade of the 5-HT_{2A} receptor has been proved in both preclinical studies (Popa et al., (2005), J. Nuerosc., 25(49): 11231-8) and clinical studies (Viola A. et al, (2002), Clin. Neurophysiol., 113(3) 429-434) to be efficacious in reducing Wake After Sleep Onset, increasing Slow Wave Sleep and Total Sleep Time therefore providing consolidation of sleep.

In a first aspect, the invention provides a compound of formula (I) or a pharmaceutically acceptable salt thereof

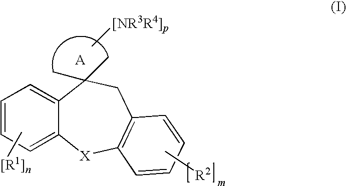

wherein

### X is CH₂, O, or S;

n is 0, 1 or 2;
m is 0, 1 or 2;
p is 0 or 1;
when present, R¹ is independently selected from the group consisting of halogen, C₁₋₃alkyl and C₁₋₃alkoxy;
when present, R² is independently selected from the group consisting of halogen, C₁₋₃alkyl and C₁₋₃alkoxy;
when p is 0, A is a spiro 5-6 membered saturated or partially unsaturated heterocyclic ring containing at least one nitrogen atom and optionally containing an additional heteroatom selected from N, S and O, the ring being optionally substituted by one or more groups independently selected from oxo and C₁₋₃alkyl;
when p is 1, A is a spiro 5-6 membered saturated or partially unsaturated carbocyclic ring;
when present, R³ and R⁴ are each independently selected from the list consisting of hydrogen and C₁₋₃alkyl; or
R³ and R⁴ together with the nitrogen to which they are attached, form a 4-6 membered saturated or partially unsaturated ring optionally containing one or more additional heteroatoms selected from O, N and S, wherein the ring is optionally substituted by one or more groups independently selected from oxo and C₁₋₃alkyl.

The term “halogen” and its abbreviation “halo” refer to fluorine, chlorine, bromine or iodine. In an embodiment unless otherwise indicated such a halo substituents is fluoro or chloro.

As used herein, a C₁₋₃alkyl substituent is a univalent radical derived by removal of a hydrogen atom from an acyclic C₁₋₃alkane. Such C₁₋₃alkyl substituents include methyl and ethyl, may be straight chain (i.e. n-propyl) or branched chain (for example isopropyl). In an embodiment, unless otherwise indicated, any C₁₋₃alkyl substituent is methyl, ethyl, n-propyl or isopropyl.

As used herein, a C₁₋₃alkoxy substituent is group of formula “R—O—” where R is C₁₋₃alkyl as defined above. Such alkoxy substituents include methoxy and ethoxy and may be straight chain (i.e. n-propoxy) or branched chain (for example isopropoxy). In an embodiment, unless otherwise indicated, any C₁₋₃alkoxy substituent is methoxy, ethoxy, n-propoxy or isopropoxy.

As used herein, the term “oxo” is the bivalent radical ═O.

In an embodiment X is CH₂ or S.

In an embodiment n is 0 or 1.

In an embodiment n is 0.

In an embodiment, when n is 1, R¹ is halogen.

In an embodiment m is 0 or 1.

In an embodiment m is 0.

In an embodiment, when m is 1, R² is halogen.

In an embodiment, when p is 1, R³ and R⁴ together with the nitrogen to which they are attached, form a 4-6 membered saturated ring optionally containing one additional heteroatom selected from O, N and S, wherein the ring is optionally substituted by one or more groups independently selected from oxo and C₁₋₃alkyl.

In a further embodiment, when p is 1, R³ and R⁴ together with the nitrogen to which they are attached, form a 5-6 membered saturated ring optionally containing one or more additional heteroatoms selected from O, N and S, wherein the ring is optionally substituted by one or more groups independently selected from oxo and C₁₋₃alkyl.

In a further embodiment, when p is 1, R³ and R⁴ together with the nitrogen to which they are attached, form a 4-5- or 6-membered ring selected from the list consisting of oxazolidinyl, morpholinyl, piperazinyl, pyrrolidinyl, piperidinyl, thiomorpholinyl and azetidinyl wherein the ring is optionally substituted by one or more groups independently selected from oxo and C₁₋₃alkyl.

In an embodiment, when p is 1, A is either cyclopentyl or cyclohexyl. In a further embodiment, A is cyclopentyl.

In an embodiment, when p is 0, A is a spiro 5-6 membered saturated heterocyclic ring containing at least one nitrogen atom and optionally containing an additional heteroatom atom selected from O, N and S, the ring being optionally substituted by one or more groups independently selected from oxo and C₁₋₃alkyl.

In a further embodiment, when p is 0, A is a spiro 5- or 6-membered saturated or partially unsaturated heterocyclic ring selected from the list consisting of oxazolidinyl, morpholinyl, piperazinyl, pyrrolidinyl, piperidinyl and thiomorpholinyl, the ring being optionally substituted by one or more groups independently selected from oxo and C₁₋₃alkyl.

In an embodiment, the compound is selected from:
N,N-dimethyl-5′,11′-dihydrospiro[cyclopentane-1,10′-dibenzo[a,d]cyclohepten]-3-amine;
4-(5′,11′-dihydrospiro[cyclopentane-1,10′-dibenzo[a,d]cyclohepten]-3-yl)morpholine;
1-(5′,11′-dihydrospiro[cyclopentane-1,10′-dibenzo[a,d]cyclohepten]-3-yl)pyrrolidine;
1-(5′,11′-dihydrospiro[cyclopentane-1,10′-dibenzo[a,d]cyclohepten]-3-yl)-4-methylpiperazine;
N-methyl-5′,11′-dihydrospiro[cyclopentane-1,10′-dibenzo[a,d]cyclohepten]-3-amine;
4-(5′,11′-dihydrospiro[cyclopentane-1,10′-dibenzo[a,d]cyclohepten]-3-yl)thiomorpholine;
1-(5′,11′-dihydrospiro[cyclopentane-1,10′-dibenzo[a,d]cyclohepten]-3-yl)azetidine;
1-(5′,11′-dihydrospiro[cyclopentane-1,10′-dibenzo[a,d]cyclohepten]-3-yl)piperidine;
N,N-dimethyl-5′,11′-dihydrospiro[cyclopentane-1,10′-dibenzo[a,d]cyclohepten]-3-amine (Isomer 1);
N,N-dimethyl-5′,11′-dihydrospiro[cyclopentane-1,10′-dibenzo[a,d]cyclohepten]-3-amine (Isomer 2);
N,N-dimethyl-5′,11′-dihydrospiro[cyclopentane-1,10′-dibenzo[a,d]cyclohepten]-3-amine (Isomer 3);
N,N-dimethyl-5′,11′-dihydrospiro[cyclopentane-1,10′-dibenzo[a,d]cyclohepten]-3-amine (Isomer 4);
4-(5′,11′-dihydrospiro[cyclopentane-1,10′-dibenzo[a,d]cyclohepten]-3-yl)morpholine (Isomer 1);
4-(5′,11′-dihydrospiro[cyclopentane-1,10′-dibenzo[a,d]cyclohepten]-3-yl)morpholine (Isomer 2);
1-(5′,11′-dihydrospiro[cyclopentane-1,10′-dibenzo[a,d]cyclohepten]-3-yl)pyrrolidine (Isomer 1);
1-(5′,11′-dihydrospiro[cyclopentane-1,10′-dibenzo[a,d]cyclohepten]-3-yl)pyrrolidine (Isomer 2);
N-Methyl-5′,11′-dihydrospiro[cyclopentane-1,10′-dibenzo[a,d]cyclohepten]-3-amine (Isomer 1);
N-Methyl-5′,11′-dihydrospiro[cyclopentane-1,10′-dibenzo[a,d]cyclohepten]-3-amine (Isomer 2);
1-(5′,11′-dihydrospiro[cyclopentane-1,10′-dibenzo[a,d]cyclohepten]-3-yl)azetidine (Isomer 1);
1-(5′,11′-dihydrospiro[cyclopentane-1,10′-dibenzo[a,d]cyclohepten]-3-yl)azetidine (Isomer 2); 5′,11′-dihydrospiro[cyclopentane-1,10′-dibenzo[a,d]cyclohepten]-3-amine;
5′,11-dihydrospiro[cyclopentane-1,10′-dibenzo[a,d]cyclohepten]-3-amine (Isomer 1);
5′,11-dihydrospiro[cyclopentane-1,10′-dibenzo[a,d]cyclohepten]-3-amine (Isomer 2);
4-(5′,11′-dihydrospiro[cyclopentane-1,10′-dibenzo[a,d]cyclohepten]-3-yl)thiomorpholine 1-oxide;
5,11-dihydro-6′H-spiro[dibenzo[a,d]cycloheptene-10,3′-piperidin]-6′-one;
5,11-dihydro-2′H-spiro[dibenzo[a,d]cycloheptene-10,4′-piperidin]-2′-one;
5,11-dihydrospiro[dibenzo[a,d]cycloheptene-10,3′-piperidine];
1′-methyl-5,11-dihydrospiro[dibenzo[a,d]cycloheptene-10,3′-piperidine];
3′,4′-dihydro-11H-spiro[dibenzo[b,f]thiepin-10,2′[1,4]oxazin]-5′(6′H)-one;
3′,4′,5′,6′-tetrahydro-11H-spiro[dibenzo[b,f]thiepin-10,2′[1,4]oxazine];
8-fluoro-3′,4′-dihydro-11H-spiro[dibenzo[b,f]thiepin-10,2′[1,4]oxazin]-5′(6′H)-one;
8-fluoro-3′,4′,5′,6′-tetrahydro-11H-spiro[dibenzo[b,f]thiepin-10,2′[1,4]oxazine]; and
1′-methyl-5,11-dihydrospiro[dibenzo[a,a]cycloheptene-10,4′-piperidine];
   or a pharmaceutically acceptable salt thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents maybe the same or different.

The compounds of formula (I) may form pharmaceutically or veterinarily acceptable salts, for example, non-toxic acid addition salts formed with inorganic acids such as hydrochloric, hydrobromic, hydroiodic, sulfuric and phosphoric acid, with carboxylic acids or with organo-sulfonic acids. Examples include the HCl, HBr, HI, sulfate or bisulfate, nitrate, phosphate or hydrogen phosphate, acetate, benzoate, succinate, saccharate, fumarate, maleate, lactate, citrate, tartrate, gluconate, camsylate, methanesulfonate, ethanesulfonate, benzenesulfonate, p-toluenesulfonate and pamoate salts. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Hereinafter, the compounds of formula (I) and their pharmaceutically acceptable salts, are referred to as “the compounds of the invention”.

It will be appreciated by those skilled in the art that certain protected derivatives of the compounds of the invention, which may be made prior to a final deprotection stage, may not possess pharmacological activity as such, but may, in certain instances, be administered orally or parenterally and thereafter metabolised in the body to form compounds defined in the first aspect which are pharmacologically active. Such derivatives may therefore be described as “prodrugs”. All protected derivatives and prodrugs of compounds defined in the first aspect are included within the scope of the invention. Examples of suitable pro-drugs for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within the compound defined in the first aspect.

The compounds of the invention may exist in solvated or hydrated form.

The compounds of the invention or solvates/hydrates of the compounds or salts, may exist in one or more polymorphic forms.

Therefore, according to a further aspect, the invention includes a solvate, hydrate or prodrug of the compounds of the invention.

Certain compounds of the invention may exist in one or more tautomeric forms. All tautomers and mixtures thereof are included in the scope of the present invention. Certain compounds of the invention possess one or more chiral centres and so exist in a number of stereoisomeric forms. Compounds having one chiral centre may exist as enantiomers or a racemic mixture containing enantiomers. Compounds having two or more chiral centres may exist as diastereoismomers or enantiomers. All sterioisomers (for example enantiomers and diastereoisomers) and mixtures thereof are included in the scope of the present invention. Racemic mixtures may be separated to give their individual enantiomer using preparative HPLC using a column with a chiral stationary phase or resolved to yield individual enantiomers utilising methods known to those skilled in the art. In addition, chiral intermediate compounds may be resolved and used to prepare individual enantiomers.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Compounds of the invention may be prepared in a variety of ways. In the following reaction schemes and hereinafter, unless otherwise stated R¹ to R⁴, A, X, n, m and p are as defined in the first aspect of the invention. These processes form further aspects of the invention.

Throughout the specification, general formulae are designated by Roman numerals (I), (II), (III), (IV) etc. Subsets of these general formulae are defined as (Ia), (Ib), (Ic), etc. . . . (IVa), (IVb), (IVc) etc.

Generally, compounds of formula (I), where A is a spiro 5-6 membered saturated or partially unsaturated carbocyclic ring and p is 1 may be prepared according to general reaction Scheme 1. Methods suitable for Scheme 1 are known in the literature (eg. J. Org. Chem. 1996, 61, 3849-3862). For example, by reacting a compound of formula (II) with a compound of formula (III) in the presence of an acid (eg glacial acetic acid) and a reducing agent (eg NaBH(OAc)₃ or NaBH₄) in a suitable solvent (eg DCE) at room temperature for 12-24 hours.

Compounds of formula (III) are either commercially available or may be prepared by procedures known to the skilled person.

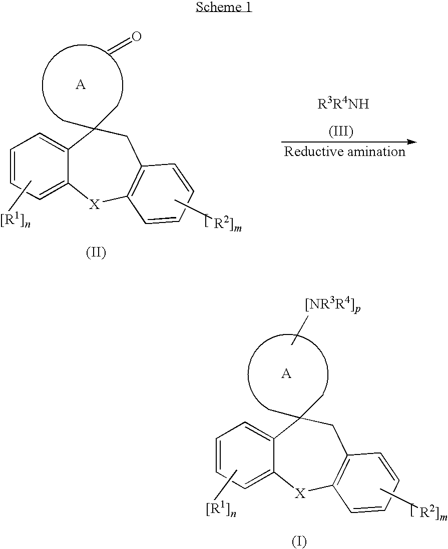

In the case of compounds of general formula (I), wherein A is a spiro 5-6 membered saturated or partially unsaturated carbocyclic ring, p is 1 and R³ and R⁴ are both hydrogen, the compounds may be prepared in two steps. Firstly, by reductive amination of a compound of formula (II) with benzylamine according to Scheme I. Secondly, by removing the benzilic protection with one of the methods described in literature (eg J. Org. Chem. 2001, 66, 5317-5328). For example by refluxing the compound with a reducing agent (eg ammonium formiate) in presence of a catalyst (eg Pd/C) in a suitable solvent (eg MeOH) for 2 hours.

In the case of compounds of general formula (I), wherein A is a spiro 5-6 membered saturated or partially unsaturated carbocyclic ring, p is 1 and R³ and R⁴ together with the nitrogen to which they are attached, form a 4-6 membered saturated or partially unsaturated ring containing a sulfinyl or a sulfonyl functional group, the compounds may be prepared in two steps. Firstly, by reductive amination of a compound of formula (II) with thiomorpholine according to Scheme I. Secondly, by oxidising the sulphur atom with one of the procedures described in literature (eg Organic Process Research & Development, 10(6), 1157-1166; 2006; PCT Int. Appl., 2007000432, 04 Jan. 2007) by reacting the compound with an oxidant (eg Oxone® solution) in a suitable solvent (eg MeOH, DCM and water) at low temperature (eg −8/10° C.) for 10 mins.

Compounds of formula (Ia), i.e. compounds of general formula (I) wherein A is a spiro 5-6 membered saturated or partially unsaturated heterocyclic ring containing at least one nitrogen atom and optionally containing an additional nitrogen or oxygen atom, the ring being substituted by oxo and p is 0, may be prepared according to reaction Scheme 2.

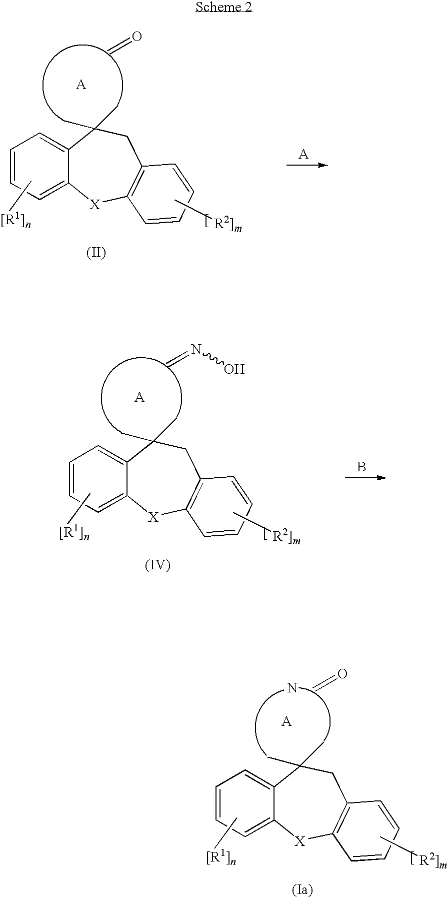

The synthesis (A) of the oxime (IV) and the Beckmann rearrangement (B) may be performed according to procedures described in literature (eg Org. Lett. 2005; 7(8), 1617-1619; J. Am. Chem. Soc. 1952, 74(10); 2680-2681). For example:

A) By reacting compounds of formula (II) with hydroxylamine hydrochloride and sodium acetate in a suitable solvent (eg MeOH, DCM and water) at room temperature for 3 days to give compounds of formula (IV);
B) By reacting compounds of formula (IV) with an acid (eg PPA) at elevated temperature (eg 100° C.) for 10 minutes.

Further compounds of general formula (I) may be prepared according to reaction Scheme 3.

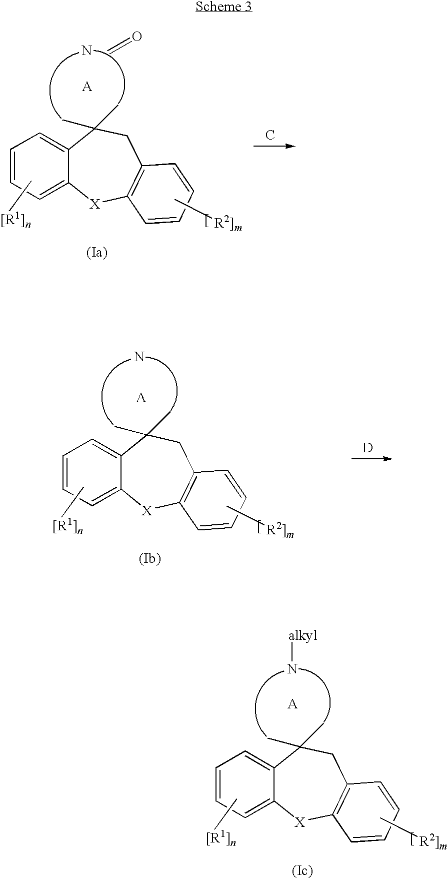

Compounds of formula (Ia), i.e. compounds of general formula (I) wherein A is a spiro 5-6 membered saturated or partially unsaturated heterocyclic ring containing at least one nitrogen atom and optionally containing an additional nitrogen or oxygen atom, the ring being substituted by oxo, and wherein p is 0, may be reduced (C) according to one of the procedures described in literature (eg Brown, H. C.; Heim, P. Selective reductions; J. Org. Chem. 1973, 38(5); 912-916) to give compounds of formula (Ib) i.e. compounds of general formula (I) wherein A is a spiro 5-6 membered saturated or partially unsaturated heterocyclic ring containing at least one nitrogen atom and optionally containing an additional nitrogen or oxygen atom and wherein p is 0. Compounds of formula (Ib) may be optionally alkylated (D) (eg J. Med. Chem. 1979, 22(7), 834-839) to give compounds of formula (Ic), i.e. compounds of general formula (I) wherein A is a spiro 5-6 membered saturated or partially unsaturated heterocyclic ring containing at least one nitrogen atom and optionally containing an additional nitrogen or oxygen atom, the ring being substituted by alkyl and wherein p is 0. For example:

C) By reacting compounds of formula (Ia) with a reducing agent (eg borane) in a suitable solvent (eg THF) at reflux for 2 hours;
D) By reacting compounds of formula (Ib) with formalin in a suitable solvent (eg formic acid) at reflux for 16-18 hours.

Alternatively, compounds of formula (Ic), i.e compounds of general formula (I) wherein A is a spiro 5-6 membered saturated or partially unsaturated heterocyclic ring containing at least one nitrogen atom and optionally containing an additional nitrogen or oxygen atom, the ring being substituted by alkyl and wherein p is 0, may be synthesised according to Scheme 4 by reducing the corresponding ketone, a compound of formula (V), whose synthesis is described in literature (eg Chemical & Pharmaceutical Bulletin, 27(9), 2056-64; 1979).

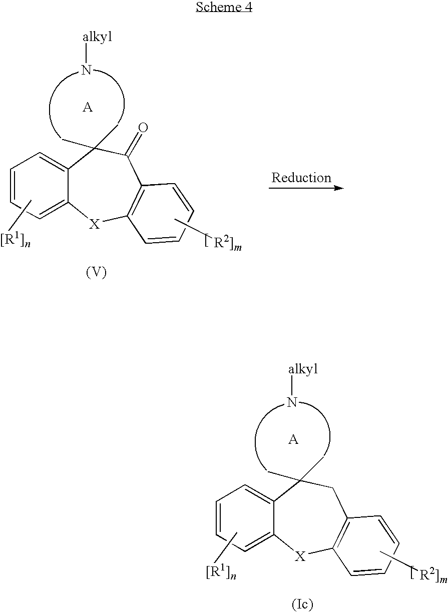

Compounds of formula (Ia), i.e. compounds of general formula (I) wherein A is a spiro 5-6 membered saturated or partially unsaturated heterocyclic ring containing at least one nitrogen atom and optionally containing an additional nitrogen or oxygen atom and wherein p is 0, may be prepared according to reaction Scheme 5.

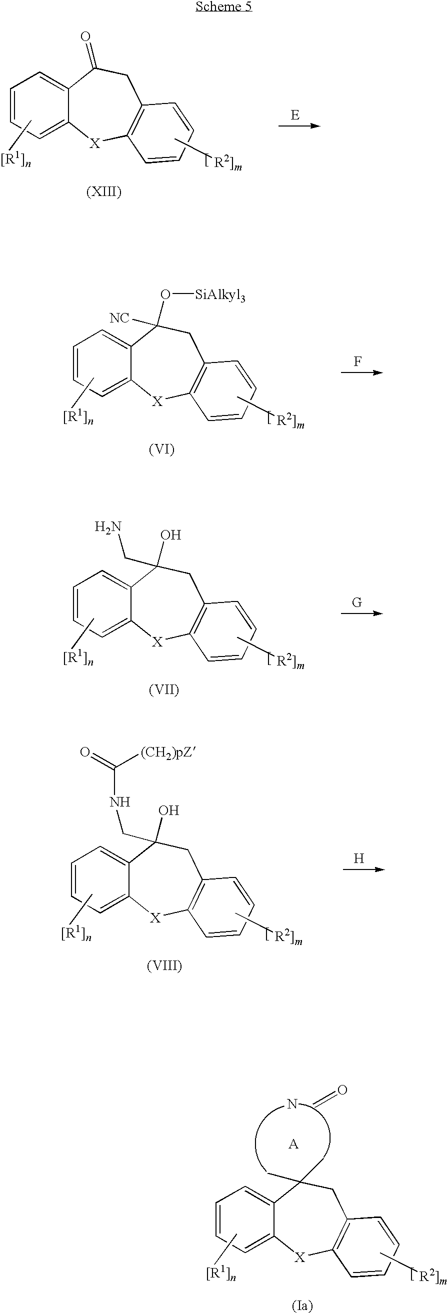

For example:

E) By reacting a compound of formula (XIII) with a suitable silyl cyande (eg trimethylsilyl cyanide) and a zinc salt (eg zinc iodide) in a suitable solvent (eg toluene) at 40° C. for 3-24 hours to give a compound of formula (VI);
F) By reacting a compound of formula (VI) with a reducing agent (eg LiAlH₄) in a suitable solvent (eg THF or diethyl ether) at reflux for 2-24 hours to give a compound of formula (VII);
G) By reacting a compound of formula (VII) with Z′—(CH₂)ₚ—CO—Z wherein Z and Z′ are leaving groups such as chlorine, bromine, etc. and wherein p is 0, 1, etc., in the presence of a suitable base (eg DIPEA, triethylamine) in a suitable solvent (eg DCM) at 0/25° C. for 2-3 hours to give a compound of formula (VIII);
H) By reacting a compound of formula (VIII) with a base (eg KOH) in a suitable solvent (eg dioxane) at reflux for 1-2 hours to give a compound of formula (Ia).

Compounds of formula (IIa), i.e compounds of general formula (II) wherein A is cyclopentyl, may be prepared according to reaction Scheme 6 by reducing compounds of formula (IX) using one of the methods well described in literature (see for example Chem. Pharm. Bull. 2004, 52 (1), 79-88). For example, by reacting a compound of formula (IX) in the presence of an acid (eg acetic acid) with a catalyst (eg Pd/C) in a suitable solvent (eg THF) at a suitable H₂ pressure (eg 4-5 atm) at room temperature for 1-4 days.

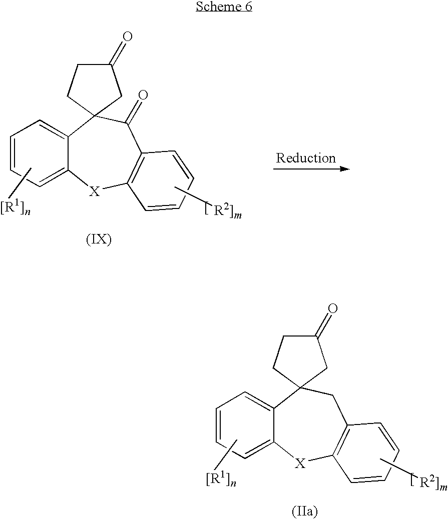

Compounds of formula (IX) may be prepared according to reaction scheme 7 by reacting the mixture of compounds of formula (X) with a suitable oxidant (eg Dess-Martin periodinane) in a suitable solvent (eg DCM) at room temperature for 3-12 hrs.

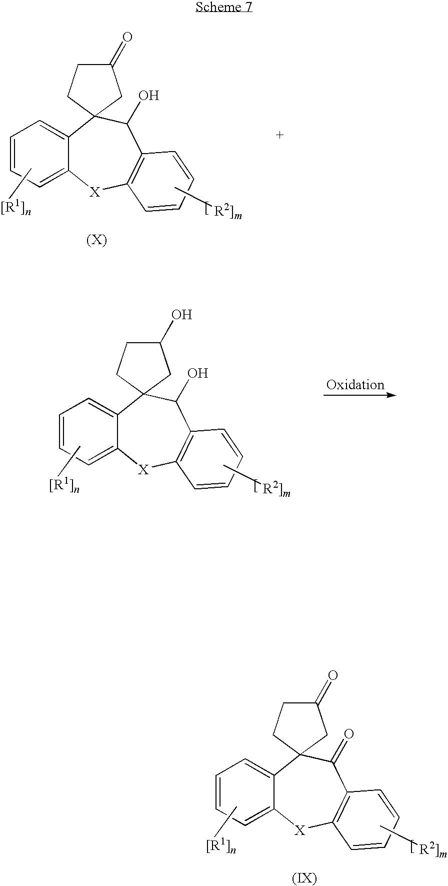

The mixture of compounds of formula (X) may be prepared according to reaction scheme 8 in 2 steps:

1. By reacting compounds of formula (XI) with borane in a suitable solvent (eg THF) at room temperature for 2-3 hours.
2. Adding water and sodium hydroxide followed by hydrogen peroxide at room temperature for 16 hours.

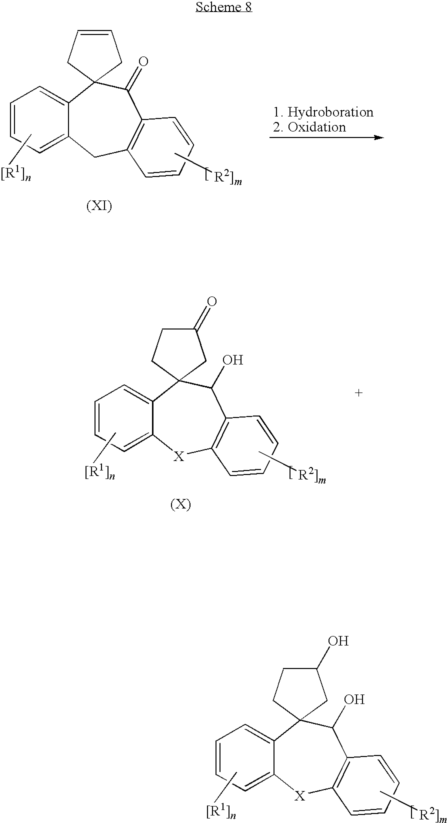

Compounds of formula (XI) may be prepared according to reaction scheme 9 by reacting compounds of formula (XII) with a suitable catalyst (eg Grubbs catalyst 2^{nd} generation) in degassed solvent (eg CH₂Cl₂) at room temperature or reflux temperature for several hours (eg 1 day).

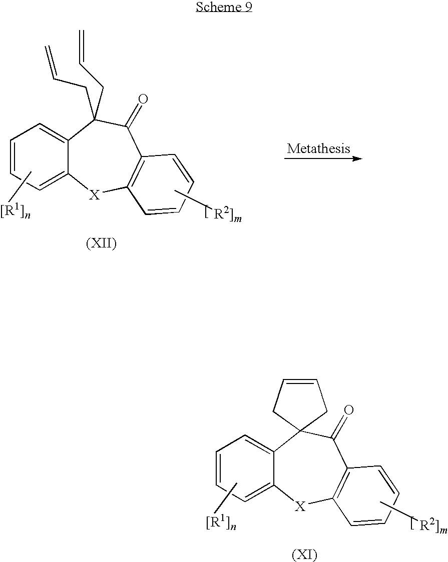

Compounds of formula (IIb), i.e. compounds of general formula (II), wherein A is cyclohexyl may be prepared according to reaction scheme 10 by esterification of the compound of formula (XIV) (see as an example Journal of the American Chemical Society, 127(50), 17877-17887; 2005), to a compound of formula (XVI), followed by cyclization to get (IIb) (see for example Bioorganic & Medicinal Chemistry Letters, 17(11), 3006-3009, 2007; Heterocycles, 65(6), 1359-1371, 2005; Synlett, (12), 2224-2226, 2004).

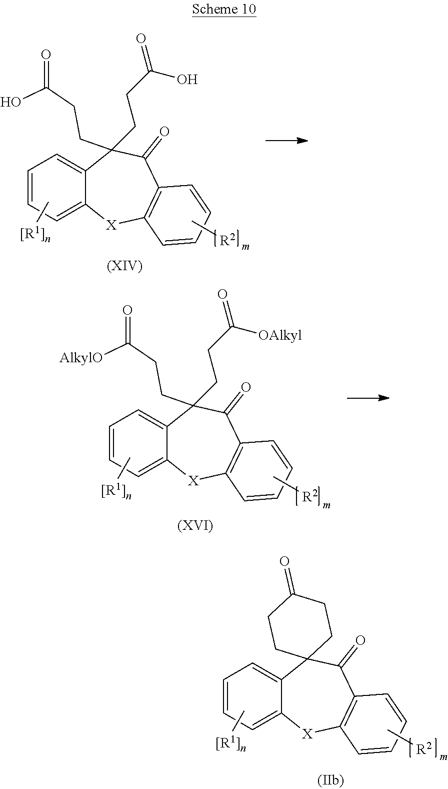

Compounds of formula (XIV) may be prepared according to reaction scheme 11 by reacting compounds of formula (XV) with an oxidant (eg Jones reagent) in a suitable solvent (eg acetone) at room temperature.

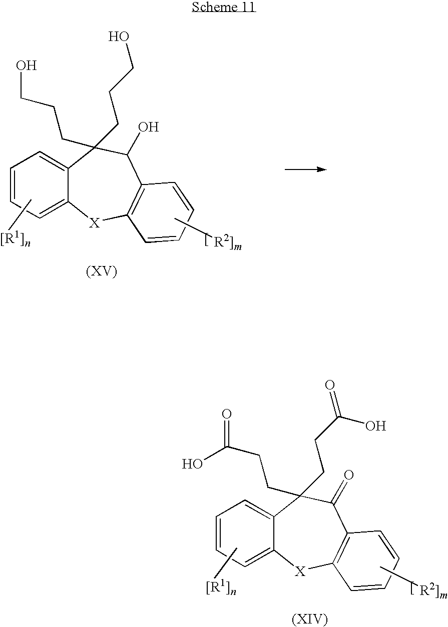

Compounds of formula (XV) may be prepared according to reaction scheme 12 by reacting compounds of formula (XII) with BH₃-THF followed by H₂O₂ oxidation in a suitable organic solvent (eg THF).

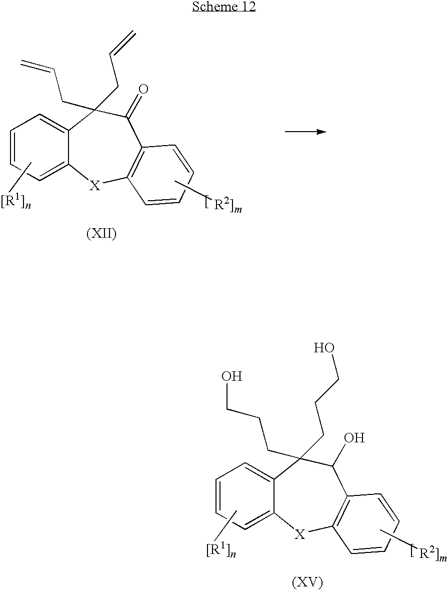

Compounds of formula (XII) may be prepared according to reaction scheme 13 by reacting compounds of formula (XIII) with a base (eg K-t-butoxide freshly prepared) and an allylating agent (eg allyl bromide) in a suitable solvent (eg t-butyl alcohol and toluene) at a moderate temperature (eg 55-60° C.) for 40-60 minutes.

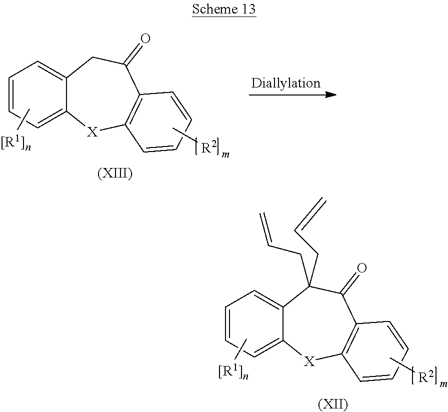

Compounds of general formula (XIII) when X is CH₂, can be prepared following a literature procedure (Journal of Medicinal Chemistry (1981), 24(9), 1021-6).

Compounds of general formula (XIII) when X is S, are either commercially available from Specs (when n, m are 0) or can be made following a literature procedure (Chemical & Pharmaceutical Bulletin (1975), 23(10), 2223-31).

Compounds of general formula (XIII) when X is O, can be made following a literature procedure (Organic Letters, 5(25), 4911-4913; 2003)

The compounds of the invention are antagonists of the H₁ receptor. In addition, some of the compounds of the invention are antagonists of the 5HT_{2A} receptor.

The compounds of the invention are useful for the treatment of diseases and conditions mediated by antagonism of the H₁ receptor and optionally by antagonism of the 5HT_{2A} receptor.

Therefore, according to an embodiment, the invention provides the compounds of the invention for use as a medicament, preferably a human medicament.

The compounds of the invention may treat diseases or conditions selected from the list consisting of: [the numbers in brackets after the listed diseases below refer to the classification code in Diagnostic and Statistical Manual of Mental Disorders, 4th Edition, published by the American Psychiatric Association (DSM-IV) and/or the International Classification of Diseases, 10th Edition (ICD-10)]:

i) Psychotic disorders for example Schizophrenia (including the subtypes Paranoid Type (295.30), Disorganised Type (295.10), Catatonic Type (295.20), Undifferentiated Type (295.90) and Residual Type (295.60)); Schizophreniform Disorder (295.40); Schizoaffective Disorder (295.70) (including the subtypes Bipolar Type and Depressive Type); Delusional Disorder (297.1) (including the subtypes Erotomanic Type, Grandiose Type, Jealous Type, Persecutory Type, Somatic Type, Mixed Type and Unspecified Type); Brief Psychotic Disorder (298.8); Shared Psychotic Disorder (297.3); Psychotic Disorder due to a General Medical Condition (including the subtypes with Delusions and with Hallucinations); Substance-Induced Psychotic Disorder (including the subtypes with Delusions (293.81) and with Hallucinations (293.82)); and Psychotic Disorder Not Otherwise Specified (298.9).

ii) Depression and mood disorders for example Depressive Episodes (including Major Depressive Episode, Manic Episode, Mixed Episode and Hypomanic Episode); Depressive Disorders (including Major Depressive Disorder, Dysthymic Disorder (300.4), Depressive Disorder Not Otherwise Specified (311)); Bipolar Disorders (including Bipolar I Disorder, Bipolar II Disorder (i.e. Recurrent Major Depressive Episodes with Hypomanic Episodes) (296.89), Cyclothymic Disorder (301.13) and Bipolar Disorder Not Otherwise Specified (296.80)); Other Mood Disorders (including Mood Disorder due to a General Medical Condition (293.83) which includes the subtypes With Depressive Features, With Major Depressive-like Episode, With Manic Features and With Mixed Features); Substance-Induced Mood Disorder (including the subtypes With Depressive Features, With Manic Features and With Mixed Features); and Mood Disorder Not Otherwise Specified (296.90).

iii) Anxiety disorders for example Social Anxiety Disorder; Panic Attack; Agoraphobia, Panic Disorder; Agoraphobia Without History of Panic Disorder (300.22); Specific Phobia (300.29) (including the subtypes Animal Type, Natural Environment Type, Blood-Injection-Injury Type, Situational Type and Other Type); Social Phobia (300.23); Obsessive-Compulsive Disorder (300.3); Posttraumatic Stress Disorder (309.81); Acute Stress Disorder (308.3); Generalized Anxiety Disorder (300.02); Anxiety Disorder Due to a General Medical Condition (293.84); Substance-Induced Anxiety Disorder; and Anxiety Disorder Not Otherwise Specified (300.00).

iv) Substance-related disorders for example Substance Use Disorders (including Substance Dependence, Substance Craving and Substance Abuse); Substance-Induced Disorders (including Substance Intoxication, Substance Withdrawal, Substance-Induced Delirium, Substance-Induced Persisting Dementia, Substance-Induced Persisting Amnestic Disorder, Substance-Induced Psychotic Disorder, Substance-Induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders (including Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-Induced Persisting Dementia, Alcohol-Induced Persisting Amnestic Disorder, Alcohol-Induced Psychotic Disorder, Alcohol-Induced Mood Disorder, Alcohol-Induced Anxiety Disorder, Alcohol-Induced Sexual Dysfunction, Alcohol-Induced Sleep Disorder and Alcohol-Related Disorder Not Otherwise Specified (291.9)); Amphetamine (or Amphetamine-Like)-Related Disorders (for example Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-Induced Anxiety Disorder, Amphetamine-Induced Sexual Dysfunction, Amphetamine-Induced Sleep Disorder and Amphetamine-Related Disorder Not Otherwise Specified (292.9)); Caffeine Related Disorders (including Caffeine Intoxication (305.90), Caffeine-Induced Anxiety Disorder, Caffeine-Induced Sleep Disorder and Caffeine-Related Disorder Not Otherwise Specified (292.9)); Cannabis-Related Disorders (including Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9)); Cocaine-Related Disorders (including Cocaine Dependence (304.20), Cocaine Abuse (305.60), Cocaine Intoxication (292.89), Cocaine Withdrawal (292.0), Cocaine Intoxication Delirium, Cocaine-Induced Psychotic Disorder, Cocaine-Induced Mood Disorder, Cocaine-Induced Anxiety Disorder, Cocaine-Induced Sexual Dysfunction, Cocaine-Induced Sleep Disorder and Cocaine-Related Disorder Not Otherwise Specified (292.9)); Hallucinogen-Related Disorders (including Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9)); Inhalant-Related Disorders (including Inhalant Dependence (304.60), Inhalant Abuse (305.90), Inhalant Intoxication (292.89), Inhalant Intoxication Delirium, Inhalant-Induced Persisting Dementia, Inhalant-Induced Psychotic Disorder, Inhalant-Induced Mood Disorder, Inhalant-Induced Anxiety Disorder and Inhalant-Related Disorder Not Otherwise Specified (292.9)); Nicotine-Related Disorders (including Nicotine Dependence (305.1), Nicotine Withdrawal (292.0) and Nicotine-Related Disorder Not Otherwise Specified (292.9)); Opioid-Related Disorders (including Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9)); Phencyclidine (or Phencyclidine-Like)-Related Disorders (including Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9)); Sedative-, Hypnotic-, or Anxiolytic-Related Disorders (including Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9)); Polysubstance-Related Disorder (including Polysubstance Dependence (304.80)); and Other (or Unknown) Substance-Related Disorders (including Anabolic Steroids, Nitrate Inhalants and Nitrous Oxide).

v) Sexual dysfunction for example Sexual Desire Disorders (including Hypoactive Sexual Desire Disorder (302.71) and Sexual Aversion Disorder (302.79)); sexual arousal disorders (including Female Sexual Arousal Disorder (302.72) and Male Erectile Disorder (302.72)); orgasmic disorders (including Female Orgasmic Disorder (302.73), Male Orgasmic Disorder (302.74) and Premature Ejaculation (302.75)); sexual pain disorder (including Dyspareunia (302.76) and Vaginismus (306.51)); Sexual Dysfunction Not Otherwise Specified (302.70); paraphilias (including Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9)); gender identity disorders (including Gender Identity Disorder in Children (302.6) and Gender Identity Disorder in Adolescents or Adults (302.85)); and Sexual Disorder Not Otherwise Specified (302.9).

vi) Sleep disorder for example primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

vii) Eating disorders such as Anorexia Nervosa (307.1) (including the subtypes Restricting Type and Binge-Eating/Purging Type); Bulimia Nervosa (307.51) (including the subtypes Purging Type and Nonpurging Type); Obesity; Compulsive Eating Disorder; Binge Eating Disorder; and Eating Disorder Not Otherwise Specified (307.50).

viii) Autism Spectrum Disorders including Autistic Disorder (299.00), Asperger's Disorder, Rett's Disorder, Childhood Disintegrative Disorder and Pervasive Developmental Disorder Not Otherwise Specified.

ix) Attention-Deficit/Hyperactivity Disorder (including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-Impulse Type (314.01) and Attention-Deficit/Hyperactivity Disorder Not Otherwise Specified (314.9)); Hyperkinetic Disorder; Disruptive Behaviour Disorders such as Conduct Disorder (including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23).

x) Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301,83), Histrionic Personality Disorder (301.50), Narcissistic Personality Disorder (301,81) Avoidant Personality Disorder (301.82), Dependent Personality Disorder (301.6), Obsessive-Compulsive Personality Disorder (301.4) and Personality Disorder Not Otherwise Specified (301.9).

xi) Enhancement of cognition including the treatment of cognition impairment in other diseases such as schizophrenia, bipolar disorder, depression, other psychiatric disorders and psychotic conditions associated with cognitive impairment, e.g. Alzheimer's disease.

In an embodiment, the invention provides the use of the compounds of the invention in the manufacture of a medicament for treating or preventing sleep disorders.

In an embodiment the sleep disorder is selected from the list consisting of: primary sleep disorders such as Dyssomnias (including Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47)); primary sleep disorders such as Parasomnias (including Nightmare Disorder (307.47), Sleep Terror Disorder (307.46), Sleepwalking Disorder (307.46) and Parasomnia Not Otherwise Specified (307.47)); Sleep Disorders Related to Another Mental Disorder (including Insomnia Related to Another Mental Disorder (307.42) and Hypersomnia Related to Another Mental Disorder (307.44)); Sleep Disorder Due to a General Medical Condition; and Substance-Induced Sleep Disorder (including the subtypes Insomnia Type, Hypersomnia Type, Parasomnia Type and Mixed Type).

The compounds of the invention may be used in combination with the following agents to treat or prevent psychotic disorders: i) antipsychotics; ii) drugs for extrapyramidal side effects, for example anticholinergics (such as benztropine, biperiden, procyclidine and trihexyphenidyl), antihistamines (such as diphenhydramine) and dopaminergics (such as amantadine); iii) antidepressants; iv) anxiolytics; and v) cognitive enhancers for example cholinesterase inhibitors (such as tacrine, donepezil, rivastigmine and galantamine).

The compounds of the invention may be used in combination with antidepressants to treat or prevent depression and mood disorders.

The compounds of the invention may be used in combination with the following agents to treat or prevent bipolar disease: i) mood stabilisers; ii) antipsychotics; and iii) antidepressants.

The compounds of the invention may be used in combination with the following agents to treat or prevent anxiety disorders: i) anxiolytics; and ii) antidepressants.

The compounds of the invention may be used in combination with the following agents to treat or prevent male sexual dysfunction: i) phosphodiesterase V inhibitors, for example vardenafil and sildenafil; ii) dopamine agonists/dopamine antagonists/dopamine transport inhibitors for example apomorphine and buproprion; iii) alpha adrenoceptor antagonists for example phentolamine; iv) prostaglandin agonists for example alprostadil; v) androgen receptor modulators such as testosterone; vi) serotonin agonists/antagonists/modulators/serotonin transporter inhibitors for example serotonin reuptake inhibitors; vii) noradrenaline transport inhibitors for example reboxetine; viii) oxytocin receptor antagonists; (ix) sodium and calcium channel inhibitors/blockers; and (x) opioid receptor antagonists.

The compounds of the invention may be used in combination with the same agents specified for male sexual dysfunction to treat or prevent female sexual dysfunction, and in addition an estrogen agonist such as estradiol.

Antipsychotic drugs include Typical Antipsychotics (for example chlorpromazine, thioridazine, mesoridazine, fluphenazine, perphenazine, prochlorperazine, trifluoperazine, thiothixine, haloperidol, molindone and loxapine); and Atypical Antipsychotics (for example clozapine, olanzapine, risperidone, quetiapine, aripirazole, ziprasidone and amisulpride).

Antidepressant drugs include serotonin reuptake inhibitors (such as citalopram, escitalopram, fluoxetine, paroxetine, sertraline femoxetine, fluvoxamine, indalpine and zimeldine); dual serotonin/noradrenaline reuptake inhibitors (such as venlafaxine, duloxetine and milnacipran); Noradrenaline reuptake inhibitors (such as reboxetine and venlafaxine); tricyclic antidepressants (such as amitriptyline, clomipramine, imipramine, maprotiline, nortriptyline and trimipramine); monoamine oxidase inhibitors (such as isocarboxazide, moclobemide, phenelzine and tranylcypromine); and others (such as bupropion, mianserin, mirtazapine, nefazodone and trazodone).

Mood stabiliser drugs include lithium, sodium valproate/valproic acid/divalproex, carbamazepine, lamotrigine, gabapentin, topiramate and tiagabine.

Anxiolytics include benzodiazepines such as alprazolam and lorazepam.

It will be appreciated that the compound of the combination or composition may be administered simultaneously (either in the same or different pharmaceutical formulations), separately or sequentially.

It will be appreciated that references herein to “treatment” extend to prophylaxis, prevention of recurrence and suppression or amelioration of symptoms (whether mild, moderate or severe) as well as the treatment of established conditions. The compound of the invention may be administered as the raw chemical but the active ingredient is suitably presented as a pharmaceutical formulation.

The compounds of the invention will normally, but not necessarily, be formulated into pharmaceutical compositions prior to administration to a patient by an appropriate route. Accordingly, in another aspect, the invention provides pharmaceutical compositions comprising a compound of the invention and one or more pharmaceutically-acceptable excipients.

As used herein, “pharmaceutically-acceptable excipient” means any pharmaceutically acceptable material present in the pharmaceutical composition or dosage form other than the compound or compounds of the invention. Typically the material gives form, consistency and performance to the pharmaceutical composition.

The pharmaceutical compositions of the invention typically contain one compound of the invention. However, in certain embodiments, the pharmaceutical compositions of the invention contain more than one compound of the invention. In addition, the pharmaceutical compositions of the invention may comprise one or more additional pharmaceutically active compounds.

Such pharmaceutical compositions of the invention may be prepared and packaged in bulk form wherein a safe and therapeutically effective amount of a compound of the invention can be dispensed and then given to the patient such as with powders or syrups. Alternatively, the pharmaceutical compositions of the invention may be prepared and packaged as dosage forms wherein each physically discrete dosage form contains a safe and effective amount of a compound of the invention. Accordingly, in another aspect, the invention provides dosage forms comprising pharmaceutical compositions of the invention.

A therapeutically effective amount of a compound of the present invention will depend upon a number of factors including, for example, the age and weight of the animal, the precise condition requiring treatment and its severity, the nature of the composition, and the route of administration, and will ultimately be at the discretion of the attendant physician or veterinarian. However, an effective amount of a compound of formula (I) for the treatment of disorders or diseases associated with H₁ antagonist activity will generally be in the range of 0.1 to 100 mg/kg body weight of recipient (mammal) per day and more usually in the range of 1 to 10 mg/kg body weight per day. Thus, for a 70 kg adult mammal, the actual amount per day would usually be from 70 to 700 mg and this amount may be given in a single dose per day or more usually in a number (such as two, three, four, five or six) of sub-doses per day such that the total daily dose is the same. An effective amount of a pharmaceutically acceptable salt thereof may be determined as a proportion of the effective amount of the compound of formula (I) per se. It is envisaged that similar dosages would be appropriate for treatment of the other conditions referred to above.

It will be recognised by one of skill in the art that the optimal quantity and spacing of individual dosages of compounds of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of compounds of the invention given per day for a defined number of days, can be ascertained by those skilled in the art using conventional course of treatment determination tests.

The compositions of the invention will typically be formulated into dosage forms which are adapted for administration to the patient by the desired route of administration. For example, dosage forms include those adapted for (1) oral administration such as tablets, capsules, caplets, pills, lozenges, powders, syrups, elixirs, suspensions, solutions, emulsions, sachets and cachets; (2) parenteral administration such as sterile solutions, suspensions, implants and powders for reconstitution; (3) transdermal administration such as transdermal patches; (4) rectal and vaginal administration such as suppositories, pessaries and foams; (5) inhalation and intranasal such as dry powders, aerosols, suspensions and solutions (sprays and drops); (6) topical administration such as creams, ointments, lotions, solutions, pastes, drops, sprays, foams and gels; (7) ocular administration such as drops, ointment, sprays, suspensions and inserts; (8) buccal and sublingual administration such as lozenges, patches, sprays, drops, chewing gums and tablets.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or portion of the body. Certain pharmaceutically-acceptable excipients may be chosen for their ability to enhance patient compliance. Certain pharmaceutically-acceptable excipients may be chosen for their ability to facilitate the release of the compound of the invention at the appropriate rate to treat the condition.

Suitable pharmaceutically-acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavouring agents, flavour masking agents, colouring agents, anticaking agents, humectants, chelating agents, plasticizers, viscosity increasing agents, rate modifying agents, antioxidants, preservatives, stabilizers, surfactants and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to determine suitable pharmaceutically-acceptable excipients in appropriate amounts for use with the compounds of the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention may be prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch and pre-gelatinized starch), gelatin, acacia, sodium alginate, alginic acid, tragacanth, guar gum, povidone, and cellulose and its derivatives (e.g. hydroxypropyl methyl cellulose). The oral solid dosage form may further comprise a disintegrant. Suitable disintegrants include starches, crospovidone, sodium starch glycolate, cros-carmellose, alginic acid, and sodium carboxymethyl cellulose. The oral solid dosage form may further comprise a lubricant. Suitable lubricants include stearic acid, magnesium stearate, calcium stearate, and sodium dodecyl sulphate. The oral solid dosage form may further comprise a glidant such as talc and colloidal silicon dioxide. The oral solid dosage form may further comprise an outer coating which may have cosmetic or functional properties.

It will be appreciated that the invention includes the following further aspects. The diseases and conditions described above extend, where appropriate, to these further aspects.
i) A compound of the invention for use in treating or preventing a disease or condition mediated by antagonism of the H₁ receptor.
ii) A method of treatment or prevention of a disease or condition mediated by antagonism of the H₁ receptor in a mammal comprising administering an effective amount of a compound of the invention.

### Supporting Compounds and Intermediates

The invention is supported by the following compounds described below.

In the procedures that follow, after each starting material, reference to an intermediate or compound is often provided. This is provided merely for assistance to the skilled chemist. The starting material may not necessarily have been prepared from the batch referred to.

Compounds are named using ACD/Name PRO6.02 chemical naming software (Advanced Chemistry Development Inc., Toronto, Ontario, M5H2L3, Canada).

Reagents were obtained from commercial suppliers (Sigma-Aldrich and Lancaster) and used without further purifications. According to standard procedures DCM and DCE were dried over calcium hydride; THF, toluene and diethyl ether were dried over Na/benzophenone and EtOH was dried over Mg/I₂ prior to use. DMF was bought already anhydrous. Anhydrous reactions were run under a positive pressure of dry N₂ or Argon.

IR spectra were recorded on a Perkin Elmer BX FT-IR system using CH₃Cl as solvent.

Thin-layer chromatography was carried out using Merck TLC plates Kieselgel 60F-254, visualised with UV light, 5% phosphomolybdic acid, aqueous potassium permanganate. Chromatographic purifications were performed on columns packed with Merck 60 silica gel, 23-400 mesh, for flash technique.

Preparative TLC was carried out using Merck pre-coated plates Kieselgel 60F-254, layer thickness 2.0 mm and layer thickness 0.20-0.25 mm.

Melting points were taken using a Gallenkamp Melting Point Apparatus and are uncorrected. Melting point determination was conducted after recrystallization of solids from appropriate solvents.

¹H-NMR were obtained at 298K, at the frequency stated using a Bruker AC200F 200 MHz or a Varian Oxford 300 MHz or a Bruker 400 Ultra Shield or a Varian INOVA 400 MHz or 500 MHz or a Bruker™ 400 MHz machine and run as a dilute solution of CDCl₃, CD₂Cl₂, CD₃OD and DMSO-d₆. All NMR spectra were reference to tetramethylsilane (TMS δ_{H} 0, δ_{c} 0). Chemical shift are expressed in parts per million (ppm, δ units). All coupling constants are reported in units of hertz (Hz), and multiplicities are labelled s (singlet), bs (broad singlet), d (doublet), t (triplet), q (quartet), dd (doublet of doublets), dt (doublet of triplets) and m (multiplet).

The purity of compounds was assessed by reversed-phase liquid chromatography and mass spectrometer (Agilent series 1100 LC/MSD) with an UV detection range of 200-245 nm and an electrospray ionization source (ESI). The LC elution method (using Zorbax Eclipse XDB, 4.6×150 mm, 5 μm C8 column) was the following: 15-35 min method at 25° C., mobile phase composed of different CH₃CN/H₂O—HCOOH 0.1% mixtures at a flow rate of 1 mL/min (all solvent were HPLC grade, Fluka). Mass spectral (MS) data were obtained using an Agilent 1100 LC/MSD VL system (G1946C) with a 0.4 mL/min flow rate using a binary solvent system of 95:5 methyl alcohol/water. UV detection was monitored at 254 nm. Mass spectra were acquired in positive mode scanning over the mass range of 50-1500. The following ion source parameters were used: drying gas flow, 10 mL/min; nebulizer pressure, 40 psig; drying gas temperature, 350° C.

Alternatively HPLC spectra were performed using a reversed-phase liquid chromatography (ProStar 210/215 PrepStar218) and UV-Vis Detector (ProStar 325) with an UV detection range of 200-245 nm. The LC elution method (using Varian Polaris 5 C-18, 150×4.6 mm) was the following: 15-35 min method at 25° C., mobile phase composed of different CH₃CN/H₂O—HCOOH 0.1% mixtures at a flow rate of 1 mL/min (all solvent were HPLC grade, Fluka).

Where required GC-MS were performed after dissolution in DCM (using GC Varian Star3400Cx-MS Varian Saturn 3) adapted for capillary column (Factor Four™, VF-5 ms, 30 m, 0.25 mm, 0.25 μm, Part NumberCP8944). Oven temperature at injection and for 20 minutes 60° C. and then a programmed temperature run at 20° C./min to 280° C., a final isothermal period of 20 min.

For the chiral separation and the chiral quality control two different techniques were used:

1) Supercritical Fluid Choromatography (SFC): analytical chromatography was performed on a Berger SFC Analytix, while for the preparative SFC, a Gilson SFC series SF3 was used.
2) High Performance Liquid Chromatography (HPLC): chiral Preparative HPLC was performed using a Waters 600 HPLC system and Agilent series 1100 instrument, while for analytical chromatography an Agilent series 1100 HPLC was used.

### Abbreviations

The following lists the abbreviations used:
NaOH sodium hydroxide
NaHCO₃ sodium bicarbonate
NaBH₄ sodium borohydride
NaBH(OAc)₃ sodium triacetoxyborohydride
KOH potassium hydroxide
KCN potassium cyanide
CHCl₃ chloroform
MeOH methanol
EtOH ethanol
DCM dichloromethane
DCE dichloroethane
THF tetrahydrofurane
DMF dimethylformamide
PPA polyphosphoric acid
EtOAc Ethyl acetate
Et₃N triethylamine
DMSO dimethyl sulfoxide
CDCl₃ chloroform-d
CD₂Cl₂ dichloromethane-d₂
CD₃OD methanol-d₄
DMSO-d₆ dimethyl sulfoxide-d₆
cHex cyclohexane
BOC₂O di-tert-butyl dicarbonate
SCX strong cation resin
TEA triethyl amine
TFA trifluoro acetic acid
AcOH acetic acid
e.e. enantiomeric excess
d.e. diastereoisomeric
excess

### Nomenclature

From reductive amination reaction (Scheme 1 in the description) of Intermediate 13 (racemate) with achiral amines, 4 products were obtained: 2 diastereoisomers (ratio usually in the range between 75/25 and 92/8) and the corresponding enantiomers.

Conventionally, the major diastereoisomer is named diastereoisomer 1 and the minor diastereoisomer is named diastereoisomer 2. Each diastereoisomer exists in the two enantiomeric forms: they are named enantiomer 1 and enantiomer 2 after the order of elution from chiral chromatography. An exemplary scheme is provided starting from Intermediate 13.

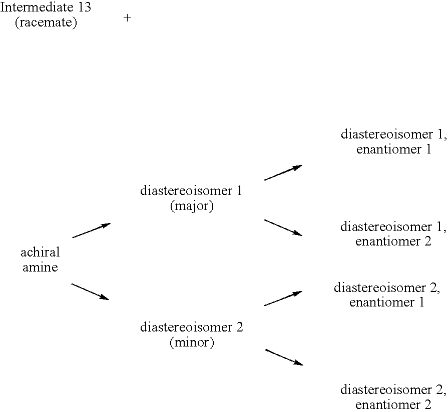

Apart from Compound 1, whose purification by chiral chromatography led to the isolation of all the four products as single enantiomers, only the enantiomers relative to the major diastereoisomer (“diastereoisomer 1, enantiomer 1” and “diastereoisomer 1, enantiomer 2”) were collected.

For the reader's benefit the term:

(diastereoisomer 1, enantiomer 1) will be named from now on isomer 1,
(diastereoisomer 1, enantiomer 2) will be named from now on isomer 2;
(diastereoisomer 2, enantiomer 1) will be named from now on isomer 3;
(diastereoisomer 2, enantiomer 2) will be named from now on isomer 4.

### Intermediate 1: Ethyl 2-(phenylmethyl)benzoate

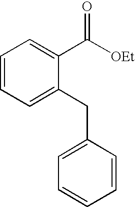

O-benzylbenzoic acid (5 g, 23.6 mmol; Sigma-Aldrich) in ethanol (50 mL) and conc. sulfuric acid (2.5 mL) was refluxed for five hours. After cooling, the major part of the solvent was evaporated to get a residue redissolved in the least amount of water and extracted using diethyl ether. The organic phase was washed using a diluted solution of NaOH, then washed with brine and dried. The organic phase was evaporated to dryness to give the title compound (4.86 g) which was used in the next step without further purification;

MS (ESI) m/z: 263 [M+Na]⁺; IR (CHCl₃) (v, cm⁻¹): 1261, 1713, 3012, 3028; ¹HNMR (CDCl₃): δ 1.28 (t, 3H), 4.26 (q, 2H), 4.37 (s, 2H), 7.10-7.44 (m, 8H), 7.85-7.89 (m, 1H).

### Intermediate 2: [2-(Phenylmethyl)phenyl]methanol

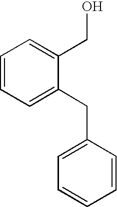

Ethyl 2-(phenylmethyl)benzoate (Intermediate 1, 4.8 g, 20 mmol) in dry ether (10 mL) was added to a solution of LiAlH₄ (0.85 g, 22 mmol) in dry ether (30 mL) under N₂ atm at 0° C. The solution was heated to reflux for 2.5 hours. After cooling, ice and diluted sulfuric acid were added and the mixture was filtered through a pad of Celite® to remove aluminum salts. The filtrate was extracted with diethyl ether and the organic layer was dried and evaporated to dryness affording the title compound as an oily product (3.96 g). It was used in the next step without further purification;

MS (ESI) m/z: 221 [M+Na]⁺; IR (CHCl₃) (v, cm⁻¹): 1217, 1453, 1494, 3013, 3608; ¹HNMR (CDCl₃): δ 4.08 (s, 2H), 4.64 (s, 2H), 7.11-7.42 (m, 9H).

### Intermediate 3: 1-(Bromomethyl)-2-(phenylmethyl)benzene

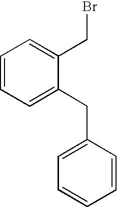

A mixture of [2-(phenylmethyl)phenyl]methanol (Intermediate 2, 3.70 g, 19 mmol) and 48% aqueous hydrobromic acid (15 mL) was refluxed for 3 hours. After cooling the reaction mixture was diluted with distilled water. The oil which was separated, was collected by dichloromethane extraction and then washed with water, dried and evaporated to give the title compound (4.53 g). It was used in the next step without further purification;

GC-MS: 181 [M−Br]+; IR (CHCl₃) (v, cm⁻¹): 1454, 1494, 3011; ¹HNMR (CDCl₃): δ 4.15 (s, 2H), 4.44 (s, 2H), 7.12-7.36 (m, 9H).

### Intermediate 4: [2-(Phenylmethyl)phenyl]acetonitrile

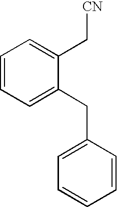

A solution of KCN (0.90 g, 14 mmol) and 1-(bromomethyl)-2-(phenylmethyl)benzene (Intermediate 3, 3.0 g, 11 mmol) in absolute ethanol (30 mL) was heated to reflux for 8 hours. After cooling the reaction mixture was diluted with water (50 mL) and then extracted with diethyl ether. The organic phase was washed with brine, dried and evaporated to give the title compound as an oil (1.95 g). It was used in the next step without further purification;

MS (ESI) m/z: 230 [M+Na]⁺; IR (CHCl₃) (v, cm⁻¹): 1454, 1494, 2252, 2926, 3024; ¹HNMR (CDCl₃): δ 3.54 (s, 2H), 4.03 (s, 2H), 7.06-7.43 (m, 9H).

### Intermediate 5: [2-(Phenylmethyl)phenyl]acetic acid

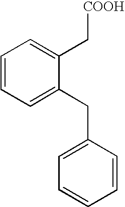

To a solution of [2-(phenylmethyl)phenyl]acetonitrile (Intermediate 4, 1.7 g, 8.04 mmol) in absolute ethanol (10 mL) was added a solution of KOH (1.22 g, 22 mmol) in distilled water (1.4 mL). The reaction mixture was heated to reflux for 20 hours. After cooling, the major part of the ethanol was evaporated and the residue was washed using DCM. The alkaline aqueous phase was acidified and extracted using DCM. The organic layer was washed with brine, dried and concentrated under reduced pressure providing the title compound (1.09 g). It was used in the next step without further purification;

MS (ESI) m/z: 249 [M+Na]⁺; IR (CHCl₃) (v, cm⁻¹): 1453, 1495, 1710, 3013, 3493; ¹HNMR (CDCl₃): δ 3.61 (s, 2H), 4.04 (s, 2H), 7.08-7.30 (m, 9H).

### Intermediate 6: 5,11-Dihydro-10H-dibenzo[a,d]cyclohepten-10-one

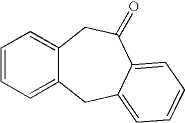

[2-(Phenylmethyl)phenyl]acetic acid (Intermediate 5, 0.100 g, 0.44 mmol) was added in small portions to PPA (0.5 mL) and stirred at 90° C. The reaction mixture was left under vigorous magnetic stirring for 30 minutes at 90° C. After cooling, water (5 mL) was added and the mixture was extracted with DCM. The organic phase was washed with saturated NaHCO₃ and dried. Evaporation of the organic solvent gave a solid residue that was recrystallized from ethanol to give the title compound (0.082 g);

MS (ESI) m/z: 231 [M+Na]⁺; IR (CHCl₃) (v, cm⁻¹): 1283, 1447, 1598, 1673, 3013; ¹HNMR (CDCl₃): δ 4.11 (s, 2H), 4.19 (s, 2H), 7.17-7.48 (m, 7H), 8.09-8.13 (m, 1H).

### Intermediate 7: 11,11-Di-2-propen-1-yl-5,11-dihydro-10H-dibenzo[a,d]cyclohepten-10-one

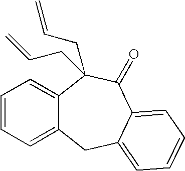

The starting material (5,11-dihydro-10H-dibenzo[a,d]cyclohepten-10-one) used in this step was derived from the combination of the previous batch (Intermediate 6) with another one prepared using the same procedure.

To a solution of K-t-butoxide, prepared by dissolving potassium metal (0.093 g, 2.4 mmol) in a mixture of t-butyl alcohol (9 mL) and dry toluene (2 mL), were added 5,11-dihydro-10H-dibenzo[a,d]cyclohepten-10-one (Intermediate 6, 0.166 g, 0.80 mmol) and allyl bromide (0.21 mL, 2.4 mmol) dropwise. When the addition was complete the reaction was left at room temperature for 1 hour, then it was heated to 50-60° C. for 45 minutes. Then it was cooled to room temperature, water was added and the mixture was extracted with diethyl ether. Recrystallization from methanol afforded the title compound (0.156 g);

MS (ESI) m/z: 311 [M+Na]⁺; IR (CHCl₃) (v, cm⁻¹): 1241, 1451, 1598, 1669, 2927, 3077;

¹HNMR (CDCl₃): δ 2.75-2.96 (m, 4H), 3.94 (s, 2H), 4.92-5.04 (m, 4H), 5.42-5.55 (m, 2H), 7.10-7.35 (m, 8H).

### Intermediate 8: Spiro[cyclopent-3-ene-1,10′-dibenzo[a,d]cyclohepten]-11′(5′H)-one

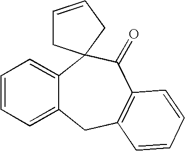

The starting material 11,11-di-2-propen-1-yl-5,11-dihydro-10H-dibenzo[a,d]cyclohepten-10-one used in this step was derived from the combination of the previous batch (Intermediate 7) with another one prepared with the same procedure.

To a solution of 11,11-di-2-propen-1-yl-5,11-dihydro-10H-dibenzo[a,d]cyclohepten-10-one (Intermediate 7, 0.300 g, 1.05 mmol) in degassed CH₂Cl₂(300 mL) was added under Argon atmosphere at room temperature Grubbs catalyst 2^{nd} generation (0.09 g, 10 mol %). After 7 hours starting material was still present (TLC check: 9/1 petroleum ether/diethyl ether), so further catalyst (0.09 g, 10 mol %) was added. The reaction mixture was left at room temperature under magnetic stirring overnight, it was then refluxed for 1 hour. After cooling the solution was absorbed on silica gel (10 eq wt relative to catalyst) and passed through a pad of silica gel, washing with 6/1 petroleum ether/diethyl ether. The resulting solution was stirred with activated charcoal (50 eq wt relative to the crude) for 12 h. The carbon was filtered off and the filtrate was concentrated in vacuo. The residue was purified by silica gel column chromatography (9/1 petroleum ether/diethyl ether) to give the title compound (0.224 g);

MS (ESI) m/z: 283 [M+Na]⁺; IR (CHCl₃) (v, cm⁻¹): 1270, 1451, 1674, 3012; ¹HNMR (CDCl₃): δ 2.92-2.99 (m, 2H), 3.56-3.63 (m, 2H), 4.36 (m, 2H), 5.71-5.74 (m, 2H), 7.09-7.51 (m, 7H), 7.78-7.88 (m, 1H).

### Intermediate 9: 3-Hydroxyspiro[cyclopentane-1,10′-dibenzo[a,d]cyclohepten]-11′(5′H)-one and Intermediate 10: 5′,11′-dihydrospiro[cyclopentane-1,10′-dibenzo[a,d]cycloheptene]-3,11′-diol

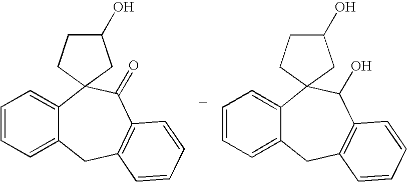

1M borane solution in THF (0.77 mL, 0.77 mmol) was added dropwise to a stirred solution of spiro[cyclopent-3-ene-1,10′-dibenzo[a,d]cyclohepten]-11′(5′H)-one (Intermediate 8, 0.200 g, 0.77 mmol) in anhydrous THF (1.6 mL) at room temperature under N₂ atmosphere. The mixture was stirred at room temperature for 2.5 hours, and then water (0.08 mL) was added dropwise, followed by 3M sodium hydroxide (0.10 mL). Hydrogen peroxide (0.12 mL, 35%) was then added at such a rate that the temperature of the mixture stayed between 30 and 50° C. After the addition was complete, the reaction mixture was stirred for 16 hours at room temperature. Diethyl ether (1.6 mL) was added to the reaction mixture and the organic phase was washed with brine and water. The organic solvent was evaporated to give a residue which was purified by silica gel column chromatography (from 3/1 to 1/1 petroleum ether/diethyl ether) to afford the title compounds.

### Intermediate 9 (0.091 g)

MS (ESI) m/z: 279 [M+1]⁺, 301 [M+Na]⁺; 261 [M−H₂O]⁺; 579 [2M+Na]⁺;

IR (CHCl₃) (v, cm⁻¹): 1451, 1596, 1670, 2929, 3011, 3612;

¹HNMR (CDCl₃): δ 1.66-1.92 (m, 2H), 2.12-2.21 (m, 1H), 2.32-2.48 (m, 1H), 2.85-2.95 (m, 1H), 3.21-3.30 (m, 1H), 4.35-4.44 (m, 3H), 7.10-7.46 (m, 7H), 7.91-7.95 (m, 1H).

### Intermediate 10 (0.079 g)

MS (ESI) m/z: 303 [M+Na]⁺;

¹HNMR (CDCl₃): δ 1.83-2.43 (m, 5H), 2.61-2.71 (m, 1H), 3.81-3.89 (m, 1H), 4.47-4.61 (m, 2H), 5.03 (s, 1H), 7.03-7.61 (m, 8H).

### Intermediate 11: 3H—Spiro[cyclopentane-1,10′-dibenzo[a,d]cycloheptene]-3,11′(5′H)-dione

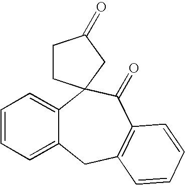

A mixture of 3-hydroxyspiro[cyclopentane-1,10′-dibenzo[a,d]cyclohepten]-11′(5′H)-one and 5′,11′-dihydrospiro[cyclopentane-1,10′-dibenzo[a,d]cycloheptene]-3,11′-diol (Intermediates 9 & 10, 0.100 g) was added to a solution of Dess-Martin periodinane (0.38 g, 0.9 mmol) in dry CH₂Cl₂ (8 mL). The reaction mixture was stirred at 25° C. for 3.5 hours. It was then diluted with DCM and washed with 1N sodium hydroxide solution. The organic phase was removed under reduced pressure and the mixture was purified by silica gel column chromatography (1/1 petroleum ether/diethyl ether) to give the title compound (0.093 g);

MS (ESI) m/z: 299 [M+Na]⁺; ¹HNMR (CDCl₃): δ 2.26-2.49 (m, 3H), 2.76-2.84 (m, 1H), 3.26-3.46 (m, 2H), 4.32-4.51 (m, 2H), 7.16-7.48 (m, 7H), 7.92-7.96 (m, 1H).

### Intermediate 12: 11′-Hydroxy-5′,11′-dihydro-3H-spiro[cyclopentane-1,10′-dibenzo[a,d]cyclohepten]-3-one and Intermediate 13: 5′,11′-dihydro-3H-spiro[cyclopentane-1,10′-dibenzo[a,d]cyclohepten]-3-one

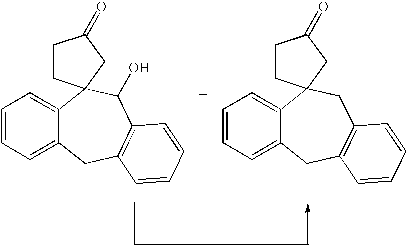

To a solution of 3H-spiro[cyclopentane-1,10′-dibenzo[a,d]cycloheptene]-3,11′(5′H)-dione (Intermediate 11, 0.092 g, 0.33 mmol) in THF (5 mL) and acetic acid (1 mL) was added Pd/C 10% (20 mg) and the reaction mixture was stirred under a hydrogen atmosphere (4 atm) for 16 hours. After this time further catalyst (Pd/C) was added (20 mg) and the reaction mixture was stirred again under a hydrogen atmosphere (4 atm) for 24 hours. Then the mixture was filtered and the solution was evaporated. The mixture was purified by preparative TLC (1/1 petroleum ether/diethyl ether) to afford the title compounds.

### Intermediate 12 (0.032 g)

MS (ESI) m/z: 301 [M+Na]⁺;

¹HNMR (CDCl₃): δ 2.27-3.10 (m, 6H), 3.67-3.75 (m, 1H), 4.48-4.57 (m, 1H), 5.49 (s, 1H), 7.05-7.59 (m, 8H).

### Intermediate 13 (0.016 g):

MS (ESI) m/z: 285 [M+Na]⁺;

¹HNMR (CDCl₃): δ 2.24-2.32 (m, 2H), 2.51-2.62 (m, 4H), 3.09-3.16 (m, 2H), 4.11-4.21 (m, 2H), 7.04-7.35 (m, 8H).

Intermediate 12 was then converted into Intermediate 13 using the typical procedure described below.

To a solution of 11′-hydroxy-5′,11′-dihydro-3H-spiro[cyclopentane-1,10′-dibenzo[a,d]cyclohepten]-3-one (Intermediate 12, 0.160 g, 0.57 mmol) in THF (18 mL) and acetic acid (4 mL) was added Pd/C 10% (160 mg) and the mixture was stirred under hydrogen atmosphere (5 atm) for 24 hours to room temperature. After this time, further catalyst (160 mg) was added and the reaction mixture was stirred under a hydrogen atmosphere (5 atm) for 24 hours. Then the mixture was filtered and the solution was evaporated. The mixture was purified by preparative TLC (1/1 petroleum ether/diethyl ether) affording Intermediate 13 (0.057 g).

### Intermediate 14 and Intermediate 15: (3E-Z)-5′,11-Dihydro-3H-spiro[cyclopentane-1,10′-dibenzo[a,d]cyclohepten]-3-one oxime

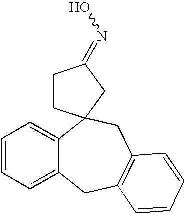

To a solution of 5′,11′-dihydro-3H-spiro[cyclopentane-1,10′-dibenzo[a,d]cyclohepten]-3-one (Intermediate 13, 0.150 g, 0.57 mmol), in MeOH (8 mL) and DCM (4 mL) was added sodium acetate (0.23 g, 2.87 mmol) and hydroxylamine hydrochloride (0.20 g, 2.86 mmol). The reaction mixture was stirred at room temperature for 72 hours and then quenched with phosphate buffer pH 7 (8 mL). The organic solvent was removed under reduced pressure and the residue was diluted with DCM (10 mL). The two phases were separated and the aqueous phase was extracted with DCM. The organic phase was evaporated to dryness and the mixture was purified by silica gel column chromatography (1/1 petroleum ether/diethyl ether) affording Intermediate 14 (0.076 g) and Intermediate 15 (0.052 g).

NMR analysis performed on the two isomers did not allow to define the geometry of the double bond. The two intermediates were combined to carry out the next step.

### Intermediate 14

MS (ESI) m/z: 278 [M+1]⁺;

¹H NMR (300 MHz, DMSO-d₆): δ 1.86-1.98 (m, 1H), 1.97-2.13 (m, 1H), 2.49 (d, 1H), 2.54-2.69 (m, 2H), 2.71 (d, 1H), 2.93 (d, 1H), 3.05 (d, 1H), 4.12 (d, 1H), 4.22 (d, 1H), 7.03-7.10 (m, 1H), 7.12-7.22 (m, 5H), 7.24-7.29 (m, 1H), 7.49 (d, 1H), 10.39 (s, 1 H).

### Intermediate 15

MS (ESI) m/z: 278 [M+1]⁺;

¹H NMR (400 MHz, DMSO-d₆): δ 1.77-2.01 (m, 2H), 2.44 (d, 1H), 2.45-2.57 (m, 1H), 2.59-2.71 (m, 1H), 2.77 (d, 1H), 2.90 (d, 1H), 3.06 (d, 1H), 4.01 (d, 1H), 4.19 (d, 1H), 6.97-7.03 (m, 1H), 7.06-7.15 (m, 5H), 7.20 (dd, 1H) 7.31 (d, 1H), 10.31 (s, 1H).

### Intermediate 16: [2-(Phenylthio)phenyl]acetic acid

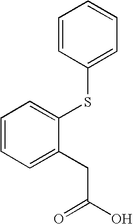

Equimolar amounts of 2-iodophenylacetic acid (200 mg, 0.763 mmol, Aldrich) and Thiophenol (78 4, 0.763 mmol, Aldrich) in a solution of KOH (475 mg) in water (5 mL) and copper bronze (5 mg, 0.0763 mmol, Aldrich) were refluxed for 5 hours. The reaction mixture was taken-up with EtOAc and it was washed with water, diluted HCl solution and brine. The aqueous phase was concentrated and filtered. 1N HCl was added to the filtered solution and a precipitate was formed. The title compound was collected after filtration as a white solid (180 mg);

MS (ESI) m/z: 267 [M+Na]⁺; ¹H NMR (200 MHz, CDCl₃): δ 3.88 (s, 2H), 7.16-7.33 (m, 7H), 7.41-7.45 (d, 2H), 10.77 (s, 1H).

Intermediate 17: Dibenzo[b,f]thiepin-10(11H)-one

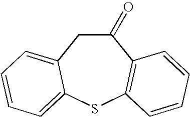

[2-(Phenylthio)phenyl]acetic acid (Intermediate 16, 100 mg, 0.410 mmol) was added portionwise to a stirred and refluxing solution of polyphosphoric acid (0.5 mL, Aldrich) in toluene (3 mL). The mixture was stirred at 110° C. for 24 hrs. After cooling, the solvent was evaporated to give a residue that was dissolved in EtOAc. The organic phase was washed with water, Na₂CO₃ aq. solution and brine and it was concentrated. The crude was purified by flash-chromatography (9/1 petroleum ether/diethyl ether) to give the title compound (30 mg);

MS (ESI) m/z: 249 [M+Na]⁺; ¹H NMR (200 MHz, CDCl₃): δ 4.34 (s, 2H), 7.12-7.44 (m, 5H), 7.55-7.62 (m, 2H), 8.15-8.20 (m, 1H).

### Intermediate 18: 10-[(Trimethylsilyl)oxy]-10,11-dihydrodibenzo[b,f]thiepin-10-carbonitrile

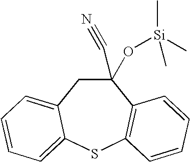

A solution of dibenzo[b,f]thiepin-10(11H)-one (Intermediate 17, 300 mg, 1.327 mmol), in anhydrous toluene (3 mL) was treated with trimethylsilyl cyanide (498 μL, 3.98 mmol, Fluka) and zinc iodide (127 mg, 0.1398 mmol, Aldrich). The mixture was stirred at 40° C. for 24 hrs. After evaporation of the solvent, the residue was purified by silica gel chromatography (from 1/0 to 95/5 petroleum ether/diethyl ether) and by preparative TLC (5/1 petroleum ether/diethyl ether) to give the title compound;

MS (ESI) m/z: 326 [M+H]⁺, 348 [M+Na]⁺; ¹H NMR (200 MHz, CDCl₃): δ 0.19 (s, 9H), 3.68 (d, 1H), 4.21 (d, 1H), 7.19-7.77 (m, 8H).

### Intermediate 19: 10-(Aminomethyl)-10,11-dihydrodibenzo[b,f]thiepin-10-ol

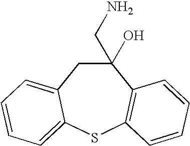

To a cooled (ice bath) suspension of LiAlH₄ powder (121 mg, 3.2 mmol, Aldrich) in anhydrous THF (1 mL) was slowly added 10-[(trimethylsilyl)oxy]-10,11-dihydrodibenzo[b,f]thiepin-10-carbonitrile (Intermediate 18, 800 mg, 2.46 mmol) and the reaction mixture was refluxed with stirring for 4 hrs. After cooling, the excess of LiAlH₄ was quenched with water and the mixture was diluted with diethyl ether and washed with 1N NaOH and brine. After evaporation of the organic layer, the crude product was purified by silica gel chromatography (from 1/0 to 98/2 EtOAc/MeOH) to give the title compound (140 mg);

MS (ESI) m/z: 258 [M+H]⁺, 280 [M+Na]⁺; ¹H NMR (500 MHz, DMSO-d₆): δ 1.51 (br.s., 2H), 2.45 (d, 1H), 2.61 (d, 1H), 3.38 (d, 1H), 3.69 (d, 1H), 5.44 (br.s., 1H), 7.12 (t, 1H), 7.17 (t, 1H), 7.23 (t, 1H), 7.29 (t, 1H), 7.37 (d, 1H), 7.50 (d, 2H), 7.64 (d, 1H).

### Intermediate 20: 2-Bromo-N-[(10-hydroxy-10,11-dihydrodibenzo[b,f]thiepin-10-yl)methyl]acetamide

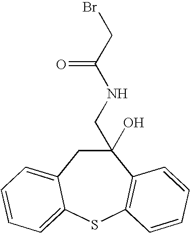

Freshly distilled DIPEA (34 μL, 0.194 mmol, Aldrich) was added to a solution of 10-(aminomethyl)-10,11-dihydrodibenzo[b,f]thiepin-10-ol (Intermediate 19, 50 mg, 0.194 mmol) in anhydrous DCM. Bromoacetyl chloride (18 μL, 0.214 mmol, Fluka) was added and the reaction mixture was stirred at room temperature for 3 hrs. EtOAc was added, the two phases were separated and the organic layer was washed with water and brine. After evaporation of the solvent, the crude product was purified by silica gel chromatography (from 1/0 to 9:1 DCM/AcOEt) to give the title compound (50 mg);

MS (ESI) m/z: 401 [M+Na]⁺; 779 [2M+Na]⁺; ¹H NMR (200 MHz, CDCl₃): δ 3.43 (d, 2H), 3.56 (d, 1H), 3.84 (d, 1H), 3.89 (s, 2H), 6.83 (s, 1H), 7.09-7.35 (m, 5H), 7.45-7.56 (m, 2H), 7.69 (d, 1H).

### Intermediate 21: {2-[(4-Fluorophenyl)thio]phenyl}acetic acid

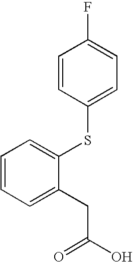

A solution of KOH (86 mg, 1.53 mmol) in water (7 mL) was reacted under N₂ atmosphere with 4-Fluorothiophenol (41 μL, 0.382 mmol, Aldrich) and the reaction mixture was stirred at 50° C. for 15 min. Then 2-iodophenylacetic acid (100 mg, 0.382 mmol, Fluka) and copper bronze (0.24 mg, 0.004 mmol, Aldrich) were added and the reaction mixture was heated at reflux overnight. The hot mixture was filtered through a fritted filter funnel and the filtrate was quenched with 1N HCl and extracted with diethyl ether. Evaporation of the solvent gave the title compound (50 mg);

MS (ESI) m/z: 285 [M+Na]⁺; IR (CHCl₃): 3066, 1713 cm⁻¹; ¹H NMR (200 MHz, CDCl₃): δ 3.87 (s, 2H), 6.91-7.00 (m, 2H), 7.18-7.36 (m, 5H), 7.85 (d, 1H), 10.22 (s, 1H).

### Intermediate 22: 8-Fluorodibenzo[b,f]thiepin-10(11H)-one

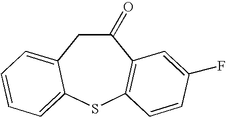

{2-[(4-fluorophenyl)thio]phenyl}acetic acid (Intermediate 21, 400 mg, 1.53 mmol) and polyphosphoric acid (4 mL) were refluxed for 2 hrs. EtOAc was added and the organic phase was extracted with water, 2M NaOH aq. solution and brine. Evaporation of the solvent gave the title compound (280 mg);

MS (ESI) m/z: 245 [M+H]⁺, 511 [2M+Na]⁺; ¹H NMR (200 MHz, CDCl₃): δ 4.36 (s, 2H), 7.06-7.24 (m, 2H), 7.30-7.44 (m, 2H), 7.53-7.63 (m, 2H), 7.84-7.90 (m, 1H).

### Intermediate 23: 8-Fluoro-10-[(trimethylsilyl)oxy]-10,11-dihydrodibenzo[b,f]thiepin-10-carbonitrile

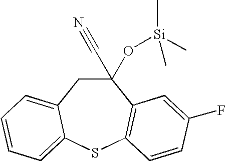

A solution of 8-fluorodibenzo[b,f]thiepin-10(11H)-one (Intermediate 22, 68 mg, 0.279 mmol) in anhydrous toluene (2 mL) was treated with trimethylsilyl cyanide (105 μL, 0.836 mmol, Fluka) and zinc iodide (27 mg, 0.084 mmol, Aldrich). The reaction mixture was stirred at 40° C. for 3.5 hr. After evaporation of the solvent, the residue was purified by preparative TLC on silica (5/1 hexane/diethyl ether) to give the title compound;

MS (ESI) m/z: 366 [M+Na]⁺.

### Intermediate 24: 10-(Aminomethyl)-8-fluoro-10,11-dihydrodibenzo[b,f]thiepin-10-ol

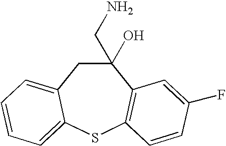

To a cooled (ice bath) suspension of LiAlH₄ powder (80 mg, 2.099 mmol, Aldrich) in anhydrous diethyl ether (2 mL) was slowly added a solution of 8-fluoro-10-[(trimethylsilyl)oxy]-10,11-dihydrodibenzo[b,f]thiepin-10-carbonitrile (Intermediate 23, 600 mg, 1.749 mmol) in anhydrous diethyl ether (3 mL) and the reaction mixture was heated at reflux for 2 hr. After cooling, excess of LiAlH₄ was quenched with water and the mixture was diluted with diethyl ether. The two phases were separated and the organic layer was washed with 15% NaOH aq. solution and brine. After evaporation of the solvent, the residue was purified by flash-chromatography on silica gel (diethyl ether and then from 1/0 to 95/5 EtOAc/MeOH) to give the title compound (220 mg);

MS (ESI) m/z: 276 [M+H]⁺, 298 [M+Na]⁺; ¹H NMR (200 MHz, CD₃OD): δ 2.61-2.76 (m, 2H), 3.38-3.46 (m, 1H), 3.80-3.90 (m, 1H), 6.80-6.91 (m, 1H), 7.11-7.18 (m, 1H), 7.22-7.30 (m, 1H), 7.38-7.51 (m, 4H).

### Intermediate 25: 2-Bromo-N-[(8-fluoro-10-hydroxy-10,11-dihydrodibenzo[b,f]thiepin-10-yl)methyl]acetamide

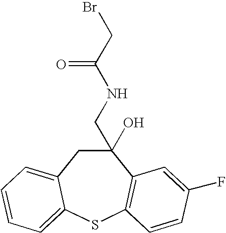

Freshly distilled DIPEA (139 μL, 0.8 mmol, Aldrich) was added to a solution of 10-(aminomethyl)-8-fluoro-10,11-dihydrodibenzo[b,f]thiepin-10-ol (Intermediate 24, 220 mg, 0.8 mmol) in anhydrous DCM (5 mL). Bromoacetyl chloride (73 4, 0.88 mmol, Fluka) was added dropwise and the reaction mixture was stirred at room temperature for 2 hr. Water was added and the mixture was extracted with EtOAc. The organic phase was washed with brine and the solvent was evaporated. The crude was purified by flash-chromatography on silica gel (diethyl ether) to give the title compound (290 mg);

¹H NMR (200 MHz, CDCl₃): δ 3.32-3.62 (m, 4H), 3.80-3.89 (m, 2H), 6.80-6.90 (m, 1H), 7.11-7.56 (m, 7H).

### Intermediate 26: 1-Methyl-4-[2-(phenylmethyl)phenyl]-4-piperidinecarbonitrile

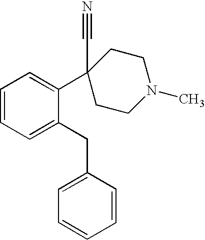

To a solution of [2-(phenylmethyl)phenyl]acetonitrile (Intermediate 4, 2.27 g, 10.96 mmol) in anhydrous DMSO (50 mL) was added NaH (1.05 g, 43.86 mmol) in small portions. After stirring the reaction at room temperature for 30 minutes, a solution of mechloroethamine hydrochloride (2.00 g, 10.42 mmol) in dry DMSO (50 mL) was added dropwise and the mixture was stirred for 1.5 hours at room temperature. The reaction mixture was then poured into ice and extracted with diethyl ether. The organic layer was evaporated and the residue was purified by silica gel column chromatography (95/3/2 ethyl acetate/methanol/triethylamine) to give the title compound (0.636 g);

MS (ESI) m/z: 291[M+1]⁺.

### Intermediate 27: 1-Methyl-4-[2-(phenylmethyl)phenyl]-4-piperidinecarboxamide and

### Intermediate 28: 1-Methylspiro[dibenzo[a,d]cycloheptene-10,4′-piperidin]-11(5H)-one

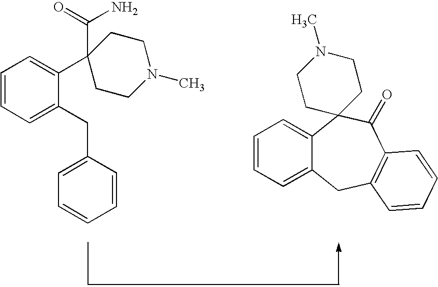

A mixture of 1-methyl-4-[2-(phenylmethyl)phenyl]-4-piperidinecarbonitrile (Intermediate 26, 0.580, 2.00 mmol) in 48% aqueous hydrobromic acid (20 mL) was heated to 110° C. for 1 day. TLC analysis (TLC check: 95/3/2 ethyl acetate/methanol/triethylamine) revealed the presence of starting material, so the reaction mixture was refluxed for 6 more days while further amounts of 48% aqueous hydrobromic acid (4×10 mL) were added. The reaction mixture was cooled down to room temperature and major amount of volatiles were removed under reduced pressure. The aqueous phase was basified with 32% NH₄OH solution and then extracted with EtOAc. The aqueous phase was then acidified to pH=5.5-5 with 1N HCl and extracted using CHCl₃. The aqueous phase was evaporated to dryness to afford a white solid dried as much as possible under high vacuum, then washed three times using CHCl₃ and filtered over filtering paper. The filtered solution was combined with the previous organic phases (CHCl₃) and evaporated under vacuum to give a residue. This solid was added to the least amount of PPA previously heated to 90-95° C. and heating was continued for 0.5 hour. After cooling, water was added and the solution was basified with 32% NH₄OH solution. The aqueous phase was then extracted with diethyl ether and the organic phase was dried and evaporated under reduced pressure. The major part of impurities were removed by silica gel column chromatography (95/3/2 ethyl acetate/methanol/Et₃N) to give an oil. The mixture was dissolved in diethyl ether and activated charcoal was added to the solution. The mixture was left under magnetic stirring at room temperature for 30 minutes. The mixture was filtered and the filtrate was evaporated to dryness to give Intermediate 27 (0.160 g) and Intermediate 28 (0.157 g).

Intermediate 27:

MS (ESI) m/z: 309 [M+1]⁺.

Intermediate 28:

MS (ESI) m/z: 292 [M+1]⁺;

¹H NMR (CDCl₃): δ 1.97-2.22 (m, 4H), 2.25 (s, 3H), 2.81-2.85 (m, 4H), 4.59 (s, 2H), 7.17-7.47 (m, 7H), 7.77-7.79 (m, 1H); ¹H NMR (500 MHz, DMSO-d₆) d ppm 1.74-1.99 (m, 4H) 2.06 (s, 3H) 2.60-2.69 (m, 4H) 4.64 (s, 2H) 7.22 (t, 1H) 7.31-7.44 (m, 5H) 7.54 (t, 1H) 7.62 (d, 1H)

Intermediate 27 was converted into Intermediate 28 using the following procedure: a mixture of Intermediate 27 (0.160 g, 0.52 mmol) in 48% aqueous hydrobromic acid (15 mL) was irradiated by microwaves in a sealed tube (1st time: T=130° C., t=20 minutes; 2nd time T=180° C., t=25 minutes). After cooling, the mixture was reduced under vacuum, the aqueous phase was basified using 32% NH₄OH solution and then extracted with EtOAc.

The aqueous phase was then acidified to pH=5.5-5 with 1N HCl and then it was extracted with CHCl₃. The aqueous phase was evaporated to dryness to give a white solid that was dried under high vacuum, washed with CHCl₃ (3×) and filtered over filtering paper. The filtered solution was combined with the previous organic phases (CHCl₃), dried and evaporated under vacuum to get a light yellow solid. The solid was dissolved in the least amount of PPA previously heated to 90/95° C. and heating was continued for 0.5 hour. After cooling, water was added and the solution was basified with 32% NH₄OH solution. The aqueous phase was then extracted using diethyl ether and the organic phase was dried and evaporated under reduced pressure. The major part of impurities were removed by silica gel column chromatography (95/3/2 ethyl acetate/methanol/Et₃N) to get an oil that was dissolved in diethyl ether. Activated charcoal was added to the solution and the mixture was left under magnetic stirring at room temperature for 30 minutes. The mixture was filtered through filtering paper and the filtrate was evaporated to dryness to give Intermediate 28 (0.039 g);

MS (ESI) m/z: 292 [M+1]⁺.

### Intermediate 29: N-(Phenylmethyl)-5′,11′-dihydrospiro[cyclopentane-1,10′-dibenzo[a,d]cyclohepten]-3-amine

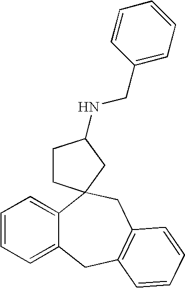

Benzylamine (0.084 mL, 0.76 mmol) and 5′,11-dihydro-3H-spiro[cyclopentane-1,10′-dibenzo[a,d]cyclohepten]-3-one (Intermediate 13, 0.10 g, 0.38 mmol) were mixed in DCE (16 mL) and glacial acetic acid (0.028 mL). After 1 hour NaBH(OAc)₃ (0.114 g, 0.054 mmol) was added. The reaction mixture was stirred for 24 hours at room temperature. Then 1N NaOH solution was added and the aqueous phase was extracted with DCM. The organic phase was dried and evaporated to dryness and the crude was purified by preparative TLC (ethyl acetate) affording the title compound (0.134 g);

MS (ESI) m/z: 354 [M+1]⁺;¹HNMR (CDCl₃): δ 1.82-2.36 (m, 6H), 3.22-3.42 (m, 2H), 3.60-3.71 (m, 1H), 3.90 (s, 2H), 4.07-4.30 (m, 2H), 7.07-7.66 (m, 13H).

### Compounds 1-8 of General Structure

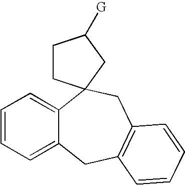

General procedure for amine synthesis: Appropriate amine (G) (1-3 eq) and 5′,11-dihydro-3H-spiro[cyclopentane-1,10′-dibenzo[a,d]cyclohepten]-3-one (Intermediate 13, 1 eq) were mixed in DCE and glacial acetic acid (1 eq). After 1-24 hours at room temperature NaBH(OAc)₃ (1.4-2.8 eq) was added and the resulting reaction mixture was stirred at room temperature from 12 to 24 hours, checking the reaction by TLC analysis. The work-up was done by adding 1N NaOH aq. and then by extracting with DCM. Products were purified by silica gel column chromatography or preparative TLC using appropriate elutent.

The use of NaBH₄ as reducing agent was necessary to get the compound N-methyl-5′,11-dihydrospiro[cyclopentane-1,10′-dibenzo[a,d]cyclohepten]-3-amine (Compound 5).

| | | TLC and | |
|---|---|---|---|
| | | column | |
| Compound | G | chromatography | ¹HNMR |
| 1: | Dimethylamine | ethyl acetate/ | ¹H NMR (300 MHz, |
| N,N-dimethyl-5′,11′- | | methanol 98/2 | DMSO-d₆): |
| dihydrospiro[cyclopentane- | | | δ 1.68-2.08 (m, |
| 1,10′-dibenzo[a,d]cyclohepten]- | | | 6 H), 2.20 (s, 6 |
| 3-amine | | | H), |
| | | | 2.77-2.93 (m, 1 H), 3.07 (d, |
| | | | 1 H), 3.19 (d, 1 |
| | | | H), 4.04 (d, 1 H), |
| | | | 4.20 (d, 1 H), |
| | | | 6.99-7.06 (m, 1 |
| | | | H), |
| | | | 7.09-7.25 (m, 6 H), |
| | | | 7.24-7.31 (m, 1 H); |
| | | | Mixture of |
| | | | diastereoisomers |
| | | | ratio ca. 8:2. |
| 2: | Morpholine | diethyl ether | ¹H NMR (500 MHz, |
| 4-(5′,11′- | | | DMSO-d₆): |
| dihydrospiro[cyclopentane- | | | δ 1.69-1.76 (m, |
| 1,10′-dibenzo[a,d]cyclohepten]- | | | 1 H), |
| 3-yl)morpholine | | | 1.76-1.85 (m, 2 H), |
| | | | 1.89-1.97 (m, 2 H), |
| | | | 1.96-2.03 (m, 1 |
| | | | H), |
| | | | 2.34-2.47 (m, 4 H), |
| | | | 2.86-2.97 (m, 1 H), |
| | | | 3.03 (d, 1 H), |
| | | | 3.20 (d, 1 H), |
| | | | 3.54-3.64 (m, 4 |
| | | | H), 3.99 (d, 1 H), |
| | | | 4.19 (d, 1 H), |
| | | | 7.00 (t, 1 H), |
| | | | 7.10 (dd, 2 H), |
| | | | 7.12-7.18 (m, 2 H), |
| | | | 7.21 (t, 2 H), |
| | | | 7.26 (d, 1 H). Mixture |
| | | | of |
| | | | diastereoisomers |
| | | | ratio ca. 7:1. |
| 3: | Pyrrolidine | ethyl acetate/ | ¹H NMR (500 MHz, |
| 1-(5′,11′- | | methanol 98/2 | DMSO-d₆): |
| dihydrospiro[cyclopentane- | | | δ 1.65-1.72 (m, |
| 1,10′-dibenzo[a,d]cyclohepten]- | | | 4 H), |
| 3-yl)pyrrolidine | | | 1.69-1.79 (m, 1 H), |
| | | | 1.82-1.95 (m, 4 H), |
| | | | 1.95-2.03 (m, 1 |
| | | | H), |
| | | | 2.38-2.48 (m, 4 H), |
| | | | 2.77-2.90 (m, 1 H), |
| | | | 3.06 (d, 1 H), |
| | | | 3.19 (d, 1 H), |
| | | | 4.01 (d, 1 H), |
| | | | 4.17 (d, 1 H), |
| | | | 6.99 (t, 1 H), |
| | | | 7.07-7.13 (m, 2 H), |
| | | | 7.13-7.17 (m, 2 |
| | | | H), |
| | | | 7.17-7.23 (m, 2 H), 7.27 (d, |
| | | | 1 H). Mixture of |
| | | | diastereoisomers |
| | | | ratio ca. 6:1. |
| 4: | Methylpiperazine | ethyl acetate/ | ¹H NMR (500 MHz, |
| 1-(5′,11′- | | methanol 98/2 | DMSO-d₆): |
| dihydrospiro[cyclopentane- | | | δ 1.65-2.03 (m, |
| 1,10′-dibenzo[a,d]cyclohepten]- | | | 6 H), |
| 3-yl)-4-methylpiperazine | | | 2.13-2.15 (m, 3 H), |
| | | | 2.15-2.68 (m, 8 H), |
| | | | 2.84-2.93 (m, 1 |
| | | | H), 3.00 (d, 1 H), |
| | | | 3.20 (d, 1 H), |
| | | | 3.98 (d, 1 H), |
| | | | 4.20 (d, 1 H), |
| | | | 6.96-7.28 (m, 8 |
| | | | H). Mixture of |
| | | | diastereoisomers |
| | | | ratio ca. 92:8 |
| 5: | Methylamine | ethyl acetate/ | ¹H NMR (500 MHz, |
| N-methyl-5′,11′- | | methanol/ | DMSO-d₆): |
| dihydrospiro[cyclopentane- | | triethylamine | δ 1.63-1.78 (m, |
| 1,10′-dibenzo[a,d]cyclohepten]- | | 95/3/2 | 2 H), |
| 3-amine | | | 1.87-1.98 (m, 2 H), |
| | | | 2.08-2.09 (m, 2 H), |
| | | | 2.28-2.31 (m, 3 |
| | | | H), |
| | | | 3.16-3.17 (m, 2 H), |
| | | | 3.28-3.34 (m, 1 H), |
| | | | 4.04-4.13 (m, 2 |
| | | | H), |
| | | | 6.94-7.27 (m, 8 H). Mixture |
| | | | of |
| | | | diatereoisomers |
| | | | ratio ca. 85:15. |
| 6: | Thiomorpholine | petroleum | ¹H NMR (500 MHz, |
| 4-(5′,11′- | | ether/diethyl | DMSO-d₆): |
| dihydrospiro[cyclopentane- | | ether 1/1 | δ 1.56-2.04 (m, |
| 1,10′-dibenzo[a,d]cyclohepten]- | | | 6 H), |
| 3-yl)thiomorpholine | | | 2.53-2.84 (m, 8 H), 3.00 (d, |
| | | | 1 H), |
| | | | 3.04-3.16 (m, 1 H), 3.18 (d, |
| | | | 1 H), 3.98 (d, 1 |
| | | | H), 4.20 (d, 1 H), |
| | | | 6.99 (t, 1 H), |
| | | | 7.07-7.18 (m, 4 H), |
| | | | 7.21 (t, 2 H), |
| | | | 7.26 (d, 1 H). Mixture |
| | | | of |
| | | | diastereoismers |
| | | | ratio ca. 90:10 |
| 7: | Azetidine | ethyl acetate/ | ¹H NMR (400 MHz, |
| 1-(5′,11′- | | methanol/ | DMSO-d₆): |
| dihydrospiro[cyclopentane- | | triethylamine | δ 1.58-1.86 (m, |
| 1,10′-dibenzo[a,d]cyclohepten]- | | 95/3/2 | 4 H), |
| 3-yl)azetidine | | | 1.83-2.06 (m, 4 H), 3.02 (t, |
| | | | 1 H), |
| | | | 3.00-3.17 (m, 4 H), 3.11 (d, |
| | | | 2 H), |
| | | | 3.99-4.20 (m, 2 H), |
| | | | 6.94-7.30 (m, 8 H). |
| | | | Mixture of |
| | | | diastereoisomers |
| | | | ratio ca. 85:15. |
| 8: | Piperidine | EtOAc/MeOH | ¹H NMR (500 MHz, |
| 1-(5′,11′- | | 98/2 | DMSO-d₆): |
| dihydrospiro[cyclopentane- | | | δ 1.35-1.43 (m, |
| 1,10′-dibenzo[a,d]cyclohepten]- | | | 2 H), |
| 3-yl)piperidine | | | 1.46-1.58 (m, 4 H), |
| | | | 1.64-1.74 (m, 1 H), |
| | | | 1.72-1.87 (m, 2 |
| | | | H), |
| | | | 1.87-2.04 (m, 3 H), |
| | | | 2.32-2.49 (m, 4 H), |
| | | | 2.87-2.97 (m, 1 |
| | | | H), 3.01 (d, 1 H), |
| | | | 3.19 (d, 1 H), |
| | | | 3.98 (d, 1 H), |
| | | | 4.20 (d, 1 H), |
| | | | 6.99 (t, 1 H), |
| | | | 7.08-7.13 (m, 2 H), |
| | | | 7.12-7.18 (m, 2 |
| | | | H), |
| | | | 7.18-7.23 (m, 2 H), 7.25 (d, |
| | | | 1 H). Mixture of |
| | | | diastereoisomers |
| | | | ratio ca. 90:10. |

### Compound 9: N,N-Dimethyl-5′,11-dihydrospiro[cyclopentane-1,10′-dibenzo[a,d]cyclohepten]-3-amine (Isomer 1)

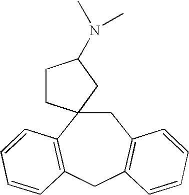

40 mg of N,N-dimethyl-5′,11′-dihydrospiro[cyclopentane-1,10′-dibenzo[a,d]cyclohepten]-3-amine (Compound 1, 0.14 mmol) were purified by chiral chromatography [SFC chromatographic conditions column: CHIRALCEL OD-H, 25×0.46 cm, pressure: 100 bar, Flow rate: 2.0 mL/min, UV detection: CD 220 nm, modifier: Ethanol+0.1% isopropylamine 10%]: Rt=11.68 min. Solvents were removed under reduced pressure to give the title compound (7.9 mg);

¹H NMR (500 MHz, DMSO-d₆): δ 1.64-2.04 (m, 6H), 2.17 (s, 6H), 2.72-2.85 (m, 1H), 3.05 (d, 1H), 3.18 (d, 1H), 4.02 (d, 1H), 4.18 (d, 1H), 6.91-7.32 (m, 8H); Chiral analysis, SFC chromatographic conditions [CHIRALCEL OD-H, 25×0.46 cm, pressure: 100 bar, Flow rate: 2.0 mL/min, UV detection: CD 220 nm, modifier: Ethanol+0.1% isopropylamine 10%]: Rt=12.42 min. (100% ee).

### Compound 10: N,N-Dimethyl-5′,11′-dihydrospiro[cyclopentane-1,10′-dibenzo[a,d]cyclohepten]-3-amine (Isomer 2)

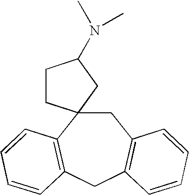

40 mg of N,N-dimethyl-5′,11′-dihydrospiro[cyclopentane-1,10′-dibenzo[a,d]cyclohepten]-3-amine (Compound 1, 0.14 mmol) were purified by chiral chromatography [SFC chromatographic conditions column: CHIRALCEL OD-H, 25×0.46 cm, pressure: 100 bar, Flow rate: 2.0 mL/min, UV detection: CD 220 nm, modifier: Ethanol+0.1% isopropylamine 10%]: Rt=13.23 min. Solvents were removed under reduced pressure to give the title compound (5.4 mg);

¹H NMR (500 MHz, DMSO-d₆): δ 1.64-2.04 (m, 6H), 2.17 (s, 6H), 2.72-2.85 (m, 1H), 3.05 (d, 1H), 3.18 (d, 1H), 4.02 (d, 1H), 4.18 (d, 1H), 6.91-7.32 (m, 8H); Chiral analysis, SFC chromatographic conditions [CHIRALCEL OD-H, 25×0.46 cm, pressure: 100 bar, Flow rate: 2.0 mL/min, UV detection: CD 220 nm, modifier: Ethanol+0.1% isopropylamine 10%]: Rt=14.05 min. (100% ee).

### Compound 11: N,N-Dimethyl-5′,11′-dihydrospiro[cyclopentane-1,10′-dibenzo[a,d]cyclohepten]-3-amine (Isomer 3)

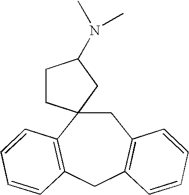

40 mg of N,N-dimethyl-5′,11′-dihydrospiro[cyclopentane-1,10′-dibenzo[a,d]cyclohepten]-3-amine (Compound 1, 0.14 mmol) were purified by chiral chromatography [SFC chromatographic conditions column: CHIRALCEL OD-H, 25×0.46 cm, pressure: 100 bar, Flow rate: 2.0 mL/min, UV detection: CD 220 nm, modifier: Ethanol+0.1% isopropylamine 10%]: Rt=14.79 min. Solvents were removed under reduced pressure to give the title compound (1.5 mg);

¹H NMR (500 MHz, DMSO-d₆): δ 1.49-2.23 (m, 12H), 2.70-2.91 (m, 1H), 2.93-3.21 (m, 2H), 3.94-4.23 (m, 2H), 6.92-7.43 (m, 8H).

### Compound 12: N,N-Dimethyl-5′,11′-dihydrospiro[cyclopentane-1,10′-dibenzo[a,d]cyclohepten]-3-amine (Isomer 4)

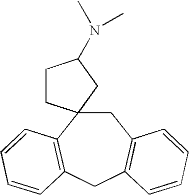

40 mg of N,N-dimethyl-5′,11′-dihydrospiro[cyclopentane-1,10′-dibenzo[a,d]cyclohepten]-3-amine (Compound 1, 0.14 mmol) were purified by chiral chromatography [SFC chromatographic conditions column: CHIRALCEL OD-H, 25×0.46 cm, pressure: 100 bar, Flow rate: 2.0 mL/min, UV detection: CD 220 nm, modifier: Ethanol+0.1% isopropylamine 10%] Rt=21.65 min. Solvents were removed under reduced pressure to give the title compound (0.7 mg);

¹H NMR (500 MHz, DMSO-d₆): δ 1.65 (dd, 1H), 1.78-1.91 (m, 3H), 2.04 (dd, 1H), 2.07-2.13 (m, 1H), 2.13-2.18 (m, 6H), 2.76-2.84 (m, 1H), 3.02-3.12 (m, 2H), 4.04-4.15 (m, 2H), 6.97-7.37 (m, 8H).

### Compound 13: 4-(5′,11′-Dihydrospiro[cyclopentane-1,10′-dibenzo[a,d]cyclohepten]-3-yl)morpholine (Isomer 1)

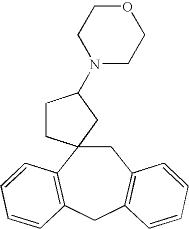

53 mg of 4-(5′,11′-dihydrospiro[cyclopentane-1,10′-dibenzo[a,d]cyclohepten]-3-yl)morpholine (Compound 2, 0.16 mmol) were purified by chiral chromatography [semipreparative chiral SFC conditions column: CHIRALCEL OJ-H, 25×2.1 cm, pressure: 176 bar, Flow rate: 22 mL/min, UV detection: CD 220 nm, modifier: Ethanol+0.1% isopropylamine 8%]. Solvents were removed under reduced pressure to give the title compound as a colourless oil (10.2 mg);

¹H NMR (400 MHz, DMSO-d₆): δ 1.64-2.10 (m, 6H), 2.30-2.58 (m, 4H), 2.88-2.99 (m, 1H), 3.01-3.12 (m, 1H), 3.16-3.27 (m, 1H), 3.51-3.71 (m, 4H), 4.00 (d, 1H), 4.21 (d, 1H), 6.94-7.36 (m, 8H); Chiral analysis, SFC chromatographic conditions [CHIRALPAK OJ-H, 25×0.46 cm, pressure: 100 bar, Flow rate: 2.0 mL/min, UV detection: CD 240 nm, modifier: Ethanol+0.1% isopropylamine 8%]: Rt=13.58 min. (100% ee).

### Compound 14: 4-(5′,11′-Dihydrospiro[cyclopentane-1,10′-dibenzo[a,d]cyclohepten]-3-yl)morpholine (Isomer 2)

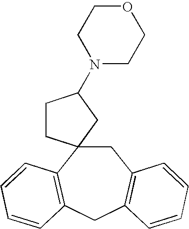

53 mg of 4-(5′,11′-dihydrospiro[cyclopentane-1,10′-dibenzo[a,d]cyclohepten]-3-yl)morpholine (Compound 2, 0.16 mmol) were purified by chiral chromatography [semipreparative chiral SFC conditions column: CHIRALCEL OJ-H, 25×2.1 cm, pressure: 176 bar, Flow rate: 22 mL/min, UV detection: CD 220 nm, modifier: Ethanol+0.1% isopropylamine 8%]. Solvents were removed under reduced pressure to give the title compound as a colourless oil (10.6 mg);

¹H NMR (400 MHz, DMSO-d₆): δ 1.62-2.10 (m, 6H), 2.30-2.49 (m, 4H), 2.88-2.99 (m, 1H), 3.01-3.12 (m, 1H), 3.16-3.27 (m, 1H), 3.51-3.71 (m, 4H), 4.00 (d, 1H), 4.21 (d, 1H), 6.94-7.36 (m, 8H); Chiral analysis, SFC chromatographic conditions [CHIRALPAK OJ-H, 25×0.46 cm, pressure: 100 bar, Flow rate: 2.0 mL/min, UV detection: CD 240 nm, modifier: Ethanol+0.1% isopropylamine 8%]: Rt=15.16 min. (100% ee).

### Compound 15: 1-(5′,11′-Dihydrospiro[cyclopentane-1,10′-dibenzo[a,d]cyclohepten]-3-yl)pyrrolidine (Isomer 1)

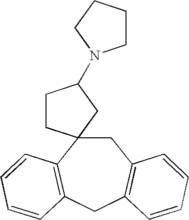

44 mg of 1-(5′,11′-dihydrospiro[cyclopentane-1,10′-dibenzo[a,d]cyclohepten]-3-yl)pyrrolidine (Compound 3, 0.14 mmol) were purified by chiral chromatography [semipreparative chiral SFC conditions column: CHIRALCEL OD-H, 25×2.1 cm, pressure: 173 bar, Flow rate: 22 mL/min, UV detection: CD 220 nm, modifier: Ethanol+0.1% isopropylamine 13%]. Solvents were removed under reduced pressure to give the title compound as a colourless oil (4.6 mg);

¹H NMR (400 MHz, DMSO-d₆): δ 1.65-1.80 (m, 5H), 1.80-2.05 (m, 5H), 2.40-2.55 (m, 4H), 2.80-2.92 (m, 1H), 3.03-3.12 (m, 1H), 3.17-3.25 (m, 1H), 4.02 (d, 1H), 4.18 (d, 1H), 6.97-7.32 (m, 8H); Chiral analysis, SFC chromatographic conditions [CHIRALCEL OD-H, 25×0.46 cm, pressure: 100 bar, Flow rate: 2.0 mL/min, UV detection: CD 240 nm, modifier: Ethanol+0.1% isopropylamine 13%]: Rt=15.40 min. (100% ee).

### Compound 16: 1-(5′,11′-Dihydrospiro[cyclopentane-1,10′-dibenzo[a,d]cyclohepten]-3-yl)pyrrolidine (Isomer 2)

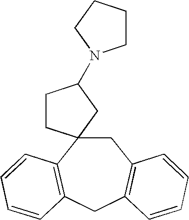

44 mg of 1-(5′,11′-dihydrospiro[cyclopentane-1,10′-dibenzo[a,d]cyclohepten]-3-yl)pyrrolidine (Compound 3, 0.14 mmol) were purified by chiral chromatography [semipreparative chiral SFC conditions column: CHIRALCEL OD-H, 25×2.1 cm, pressure: 173 bar, Flow rate: 22 mL/min, UV detection: CD 220 nm, modifier: Ethanol+0.1% isopropylamine 13%]. Solvents were removed under reduced pressure to give the title compound as a colourless oil (6.7 mg);

¹H NMR (400 MHz, DMSO-d₆): δ 1.65-1.80 (m, 5H), 1.80-2.05 (m, 5H), 2.40-2.55 (m, 4H), 2.80-2.92 (m, 1H), 3.03-3.12 (m, 1H), 3.17-3.25 (m, 1H), 4.02 (d, 1H), 4.18 (d, 1H), 6.97-7.32 (m, 8H); Chiral analysis, SFC chromatographic conditions [CHIRALCEL OD-H, 25×0.46 cm, pressure: 100 bar, Flow rate: 2.0 mL/min, UV detection: CD 240 nm, modifier: Ethanol+0.1% isopropylamine 13%]: Rt=16.75 min. (81% ee).

### Compound 17: N-Methyl-5′,11′-dihydrospiro[cyclopentane-1,10′-dibenzo[a,d]cyclohepten]-3-amine (Isomer 1)

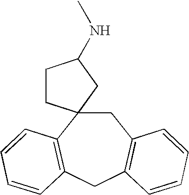

40 mg of N-methyl-5′,11′-dihydrospiro[cyclopentane-1,10′-dibenzo[a,d]cyclohepten]-3-amine (Compound 5, 0.14 mmol) were purified by chiral chromatography [semipreparative chiral SFC conditions column: CHIRALPAK AD-H, 25×2.1 cm, pressure: 180 bar, Flow rate: 22 mL/min, UV detection: CD 220 nm, modifier: Ethanol+0.1% isopropylamine 13% ]. Solvents were removed under reduced pressure to give the title compound (6.2 mg) as a colourless oil;

¹H NMR (500 MHz, DMSO-d₆): δ 1.50-2.20 (m, 6H), 2.26-2.33 (m, 3H), 3.11-3.26 (m, 2H), 3.26-3.43 (m, 1H), 4.03-4.16 (m, 2H), 6.90-7.40 (m, 8H); Chiral analysis, SFC chromatographic conditions [CHIRALPAK AD-H, 25×0.46 cm, pressure: 100 bar, Flow rate: 2.0 mL/min, UV detection: CD 225 nm, modifier: 2-Propanol+0.1% isopropylamine 13%]: Rt=12.37 min. (100& ee).

### Compound 18: N-Methyl-5′,11′-dihydrospiro[cyclopentane-1,10′-dibenzo[a,d]cyclohepten]-3-amine (Isomer 2)

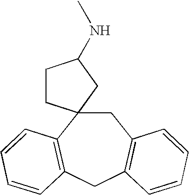

40 mg of N-methyl-5′,11′-dihydrospiro[cyclopentane-1,10′-dibenzo[a,d]cyclohepten]-3-amine (Compound 5, 0.14 mmol) were purified by chiral chromatography [semipreparative chiral SFC conditions column: CHIRALPAK AD-H, 25×2.1 cm, pressure: 180 bar, Flow rate: 22 mL/min, UV detection: CD 220 nm, modifier: Ethanol+0.1% isopropylamine 13% ]. Solvents were removed under reduced pressure to give the title compound (7.3 mg) as a colourless oil;

¹H NMR (500 MHz, DMSO-d₆): δ 1.50-2.20 (m, 6H), 2.26-2.33 (m, 3H), 3.11-3.26 (m, 2H), 3.26-3.43 (m, 1H), 4.03-4.16 (m, 2H), 6.90-7.40 (m, 8H); Chiral analysis, SFC chromatographic conditions [CHIRALPAK AD-H, 25×0.46 cm, pressure: 100 bar, Flow rate: 2.0 mL/min, UV detection: CD 225 nm, modifier: Ethanol+0.1% isopropylamine 13% ]: Rt=14.63 min. (100% ee).

Compound 19: 1-(5′,11-Dihydrospiro[cyclopentane-1,10′-dibenzo[a,d]cyclohepten]-3-yl)azetidine (Isomer 1)

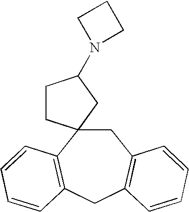

30 mg of 1-(5′,11′-dihydrospiro[cyclopentane-1,10′-dibenzo[a,d]cyclohepten]-3-yl)azetidine (Compound 7, 0.1 mmol) were purified by chiral chromatography [chromatographic conditions column: CHIRALCEL OD-H, 25×0.46 cm, n-Hexane/Ethanol 95/5% v/v, Flow rate: 0.8 mL/min, UV detection: CD 225 nm]. Solvents were removed under reduced pressure to give the title compound (5.1 mg) as a colourless oil;

¹H NMR (400 MHz, DMSO-d₆): δ 1.62-1.81 (m, 4H), 1.85-2.00 (m, 4H), 2.93-3.14 (m, 5H), 3.15-3.26 (m, 2H), 4.01-4.15 (m, 2H), 6.92-7.34 (m, 8H); Chiral analysis, SFC chromatographic conditions [CHIRALCEL OD-H, 25×0.46 cm, n-Hexane/Ethanol 95/5% v/v, Flow rate: 0.8 mL/min, UV detection: CD 225 nm]: Rt=6.2 min. (100% ee).

### Compound 20:1-(5′,11′-Dihydrospiro[cyclopentane-1,10′-dibenzo[a,d]cyclohepten]-3-yl)azetidine (Isomer 2)

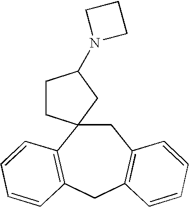

30 mg of 1-(5′,11′-dihydrospiro[cyclopentane-1,10′-dibenzo[a,d]cyclohepten]-3-yl)azetidine (Compound 7, 0.1 mmol) was purified by chiral chromatography [chromatographic conditions column: CHIRALCEL OD-H, 25×0.46 cm, n-Hexane/Ethanol 95/5% v/v, Flow rate: 0.8 mL/min, UV detection: CD 225 nm]. Solvents were removed under reduced pressure to give the title compound (6.6 mg) as a colourless oil;

¹H NMR (400 MHz, DMSO-d₆): δ 1.56-1.83 (m, 4H), 1.83-2.06 (m, 4H), 2.86-3.25 (m, 7H), 3.98-4.19 (m, 2H), 6.91-7.33 (m, 8H), Chiral analysis, SFC chromatographic conditions [CHIRALCEL OD-H, 25×0.46 cm, n-Hexane/Ethanol 95/5% v/v, Flow rate: 0.8 mL/min, UV detection: CD 225 nm]: Rt=6.9 min. (100% ee).

### Compound 21: 5′,11-Dihydrospiro[cyclopentane-1,10′-dibenzo[a,d]cyclohepten]-3-amine

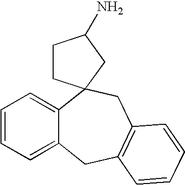

A solution of N-(phenylmethyl)-5′,11-dihydrospiro[cyclopentane-1,10′-dibenzo[a,d]cyclohepten]-3-amine (Intermediate 29, 0.090 g, 0.25 mmol), Pd/C 10% (0.018 g) and ammonium formiate (0.080 g, 1.27 mmol) in methanol (1 mL) and ethyl acetate (0.50 mL) was heated to reflux for 2 hours. Then the reaction mixture was diluted with ethyl acetate, filtered through a pad of Celite® and evaporated to dryness. The crude was purified by silica gel column chromatography (95/3/2 ethyl acetate/methanol/triethylamine) to give the title compound (0.033 g).

MS (ESI) m/z: 264 [M+1]⁺; ¹H NMR (500 MHz, DMSO-d₆): δ 1.92 (br. s., 2H), 1.58-1.75 (m, 2H), 1.84-2.06 (m, 4H), 3.16 (d, 1H), 3.22 (d, 1H), 3.58-3.68 (m, 1H), 4.02-4.15 (m, 2H), 6.98 (t, 1H), 7.06-7.17 (m, 4H), 7.17-7.28 (m, 3H).

### Compound 22: 5′,11′-Dihydrospiro[cyclopentane-1,10′-dibenzo[a,d]cyclohepten]-3-amine (Isomer 1)

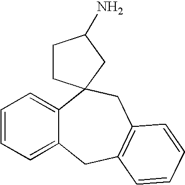

30 mg of 5′,11′-dihydrospiro[cyclopentane-1,10′-dibenzo[a,d]cyclohepten]-3-amine (Compound 21, 0.11 mmol) were purified by chiral chromatography [SFC chromatographic conditions column: CHIRALPAK AS-H, 25×2.1 cm, pressure: 162 bar, Flow rate: 22 mL/min, UV detection: CD 220 nm, modifier: 2-Propanol+0.1% isopropylamine 17%]. Solvents were removed under reduced pressure to give the title compound (1.6 mg) as a white solid;

¹H NMR (500 MHz, DMSO-d₆): δ 1.40-2.30 (m, 8H), 3.10-3.50 (m, 2H), 3.56-3.69 (m, 1H), 4.02-4.24 (m, 2H), 6.90-7.41 (m, 8H); Chiral analysis, SFC chromatographic conditions [CHIRALPAK AS-H, 25×0.46 cm, pressure: 100 bar, Flow rate: 2.0 mL/min, UV detection: CD 225 nm, modifier: 2-Propanol+0.1% isopropylamine 17%]: Rt=10.47 min; (100% e.e.).

### Compound 23: 5′,11-Dihydrospiro[cyclopentane-1,10′-dibenzo[a,d]cyclohepten]-3-amine (Isomer 2)

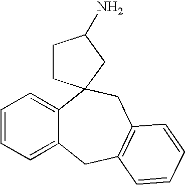

30 mg of 5′,11′-dihydrospiro[cyclopentane-1,10′-dibenzo[a,d]cyclohepten]-3-amine (Compound 21, 0.11 mmol) were purified by chiral chromatography [SFC chromatographic conditions column: CHIRALPAK AS-H, 25×2.1 cm, pressure: 162 bar, Flow rate: 22 mL/min, UV detection: CD 220 nm, modifier: 2-Propanol+0.1% isopropylamine 17%]. Solvents were removed under reduced pressure to give the title compound (3.7 mg) as a white solid;

¹H NMR (500 MHz, DMSO-d₆): δ 1.44-2.30 (m, 8H), 3.10-3.50 (m, 2H), 3.56-3.69 (m, 1H), 4.04-4.26 (m, 2H), 6.91-7.41 (m, 8H); Chiral analysis, SFC chromatographic conditions [CHIRALPAK AS-H, 25×0.46 cm, pressure: 100 bar, Flow rate: 2.0 mL/min, UV detection: CD 225 nm, modifier: 2-Propanol+0.1% isopropylamine 17%]: Rt=11.66 min; (51% e.e).

### Compound 24: 4-(5′,11′-Dihydrospiro[cyclopentane-1,10′-dibenzo[a,d]cyclohepten]-3-yl)thiomorpholine 1-oxide

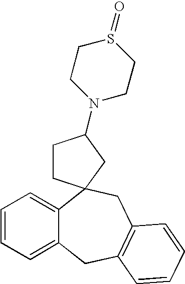

4-(5′,11′-dihydrospiro[cyclopentane-1,10′-dibenzo[a,d]cyclohepten]-3-yl)thiomorpholine (Compound 6, 0.044 g, 0.13 mmol) was dissolved in MeOH (2 mL) and DCM (2 mL) and the solution was cooled down to −8° C. The resulting slurry solution was treated with a solution of Oxone® (0.15 g, 0.25 mmol) in distilled water (2 mL) in a slow stream while the temperature was maintained below 10° C. The reaction mixture was stirred for 10 mins at −10° C. and then it was quenched using 5N NaOH solution (12 mL). The aqueous phase was extracted with DCM. The organic phase was evaporated to dryness and the crude was purified by preparative TLC (95/5 dichloromethane/methanol) to give the title compound (0.032 g). The sample which was tested was actually a recovered product from a failed reaction;

MS (ESI) m/z: 366 [M+1]⁺, 388 [M+Na]⁺; ¹H NMR (500 MHz, DMSO-d₆) δ 1.62-2.11 (m, 6H), 2.63-2.82 (m, 4H), 2.85-2.97 (m, 4H), 3.02 (d, 1H), 3.15-3.23 (m, 1H), 3.19 (d, 1H), 3.99 (d, 1H), 4.19 (d, 1H), 7.00 (t, 1H), 7.07-7.19 (m, 4H), 7.21 (t, 2H), 7.28 (d, 1 H).

### Compound 25: 5,11-dihydro-6′H-spiro[dibenzo[a,d]cycloheptene-10,3′-piperidin]-6′-one and Compound 26: 5,11-dihydro-2′H-spiro[dibenzo[a,d]cycloheptene-10,4′-piperidin]-2′-one

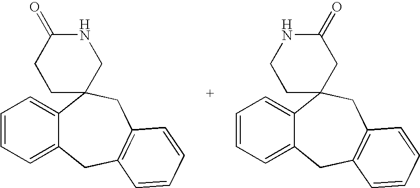

A mixture of the two isomers 3E and 3Z-5′,11′-dihydro-3H-spiro[cyclopentane-1,10′-dibenzo[a,d]cyclohepten]-3-one oxime (Intermediates 14 & 15, 0.067 g, 0.24 mmol) and PPA (1.34 mL) was heated at 100° C. for 10 minutes. After cooling to room temperature, cold water was added and the homogeneous solution was extracted using DCM. The combined organic phases were dried and concentrated. The crude product was purified by preparative TLC (1/2 petroleum ether/ethyl acetate) to give the two title compounds: 5,11-dihydro-6′H-spiro[dibenzo[a,d]cycloheptene-10,3′-piperidin]-6′-one and 5,11-dihydro-2′H-spiro[dibenzo[a,d]cycloheptene-10,4′-piperidin]-2′-one.

### Compound 25 (0.015 g)

MS (ESI) m/z: 300 [M+Na]⁺, 577 [2M+Na]⁺;

¹H NMR (500 MHz, DMSO-d₆): δ 1.62-1.71 (m, 1H), 2.15-2.37 (m, 2H), 2.38-2.50 (m, 1H), 2.87 (dd, 1H), 3.17-3.26 (m, 2H), 3.47 (d, 1H), 4.08 (d, 1H), 4.20 (d, 1H), 7.07 (t, 1H), 7.11-7.18 (m, 3H), 7.19 (d, 1H), 7.23 (t, 2H), 7.48 (d, 1H), 7.62 (d, 1H).

### Compound 26 (0.019 g)

MS (ESI) m/z: 300 [M+Na]⁺, 577 [2M+Na]⁺.

¹H NMR (500 MHz, DMSO-d₆): δ 1.50-1.74 (m, 1H), 1.95-2.72 (m, 3H), 2.96-3.60 (m, 4H), 3.94-4.32 (m, 2H), 7.00-7.70 (m, 9H). The sample consists of a mixture of the target compound (−80% mol) and of the other regioisomer compound 25 (−20% mol).

### Compound 27: 5,11-Dihydrospiro[dibenzo[a,d]cycloheptene-10,3′-piperidine]

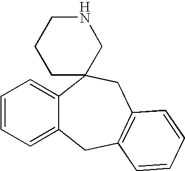

To 5,11-dihydro-6′H-spiro[dibenzo[a,d]cycloheptene-10,3′-piperidin]-6′-one (Compound 25, 0.018 g, 0.066 mmol) was added a solution of 1M borane THF complex (0.066 mL) and the reaction mixture was heated at reflux for 2 hours. After cooling, water was added dropwise and the reaction mixture was stirred for 10 minutes at room temperature. The aqueous phase was extracted with DCM. The organic phase was evaporated under reduced pressure and the residue was purified by preparative TLC (95/3/2 ethyl acetate/methanol/triethylamine) affording the title compound (2.3 mg);

MS (ESI) m/z: 264 [M+1]⁺; ¹H NMR (500 MHz, DMSO-d₆): δ 1.18-1.30 (m, 1H), 1.41 (d, 1H), 1.55 (d, 1H), 1.75-1.99 (m, 1H), 2.61 (d, 1H), 2.68 (t, 1H), 2.91 (d, 1H), 3.06 (d, 1H), 3.24-3.50 (m, 2H), 4.11 (s, 2H), 7.03 (t, 1H), 7.06-7.18 (m, 4H), 7.20 (d, 1H), 7.30 (d, 1H), 7.51 (d, 1H).

### Compound 28: 1-Methyl-5,11-dihydrospiro[dibenzo[a,d]cycloheptene-10,3′-piperidine]

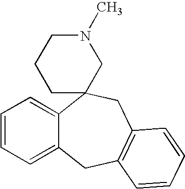

5,11-Dihydrospiro[dibenzo[a,d]cycloheptene-10,3′-piperidine] (Compound 27, 10 mg, 0.04 mmol) was dissolved in formic acid (1 mL) and the reaction mixture was refluxed for 2 hrs. Formalin (37% HCHO in H₂O, 1 mL) was added and the resulting mixture was stirred and refluxed for 16 hrs. The excess of reagents were removed under vacuum, water was added to the residue and it was basified with diluted NaOH solution. The mixture was extracted using DCM, the organic phase was washed with water and finally it was evaporated to dryness. The crude was purified by preparative TLC (95/3/2 ethyl acetate/methanol/triethylamine) affording the title compound (2.1 mg);

MS (ESI) m/z: 278 [M+1]⁺; ¹H NMR (500 MHz, DMSO-d₆): δ 1.44-1.63 (m, 2H), 1.58-1.73 (m, 1H), 1.89-2.07 (m, 2H), 2.10 (d, 1H), 2.14 (s, 3H), 2.40 (d, 1H), 2.87 (d, 1H), 3.36 (d, 1H), 3.47 (d, 1H), 4.04-4.18 (m, 2H), 7.01-7.23 (m, 7H), 7.47 (d, 1H).

### Compound 29: 3′,4′-Dihydro-11H-spiro[dibenzo[b,f]thiepin-10,2′-[1,4]oxazin]-5′(6′H)-one

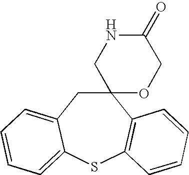

A solution of KOH (74 mg, 1.32 mmol) in water (0.1 mL) was added to a solution of 2-bromo-N-[(10-hydroxy-10,11-dihydrodibenzo[b,f]thiepin-10-yl)methyl]acetamide (Intermediate 20, 50 mg, 0.132 mmol) in dioxane (3 mL). The resulting mixture was heated at refluxing temperature for 2 hrs. After evaporation of the solvent, the crude product was diluted with EtOAc and washed with water and brine. The organic phase was evaporated to dryness and the residue was purified by preparative TLC on silica (98/2 EtOAc/MeOH) to give the title compound (13 mg);

MS (ESI) m/z: 298 [M+H]⁺; 320 [M+Na]⁺; 617 [2M+Na]⁺; ¹H NMR (500 MHz, DMSO-d₆): δ 2.94 (dd, 1H), 3.41 (d, 1H), 3.62 (d, 1H), 3.66 (d, 1H), 4.29 (d, 1H), 4.37 (d, 1H), 7.20-7.26 (m, 2H), 7.28 (t, 1H), 7.36 (t, 1H), 7.43 (d, 2H), 7.56 (d, 1H), 7.72 (d, 1H), 8.19-8.25 (m, 1H).

Compound 30: 3′,4′,5′,6′-tetrahydro-11H-spiro[dibenzo[b,f]thiepin-10,2′-[1,4]oxazine]

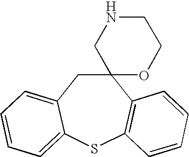

To a stirred and cooled (ice bath) suspension of LiAlH₄ (20 mg, 0.525 mmol, Aldrich) in anhydrous THF (1 mL) was added dropwise a solution of 3′,4′-dihydro-11H-spiro[dibenzo[b,f]thiepin-10,2′]1,4]oxazin]-5′(6′H)-one (Compound 29, 130 mg, 0.438 mmol) in anhydrous THF (2 mL). The reaction mixture was heated to reflux for 3 hrs. After cooling, water was added and the mixture was extracted with ethyl acetate. After evaporation of the organic layer, the residue was purified by silica gel chromatography (from 1/0 to 95/5 EtOAc/MeOH) to give the title compound (50 mg);

MS (ESI) m/z: 306 [M+Na]⁺; ¹H NMR (500 MHz, DMSO-d₆): δ 2.57 (d, 1H), 2.72 (d, 1H), 2.86 (d, 2H), 3.62 (d, 1H), 3.80-3.92 (m, 2H), 4.09-4.21 (m, 1H), 7.15 (t, 1H), 7.19 (t, 1H), 7.25 (t, 1H), 7.32 (t, 1H), 7.37 (d, 1H), 7.52 (d, 1H), 7.57 (d, 1H), 7.79 (d, 1H).

### Compound 31: 8-Fluoro-3′,4′-dihydro-11H-spiro[dibenzo[b,f]thiepin-10,2′-[1,4]oxazin]-51(61H)-one

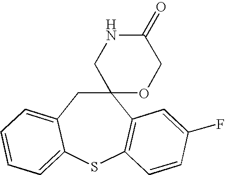

A solution of KOH (411 mg, 7.32 mmol) in water (1 mL) was added to a solution of 2-bromo-N-[(8-fluoro-10-hydroxy-10,11-dihydrodibenzo[b,f]thiepin-10-yl)methyl]acetamide (Intermediate 25, 290 mg, 0.73 mmol) in dioxane (10 mL). The resulting mixture was heated to reflux for 1 hr. After evaporation of the solvents, water was added and a precipitate was formed. The solid was collected after filtration, washing with water, and it was purified by silica gel chromatography (EtOAc) to give the title compound (150 mg);

MS (ESI) m/z: 316 [M+H]⁺; ¹H NMR (500 MHz, DMSO-d₆): δ 2.98 (dd, 1H), 3.43 (d, 1H), 3.64 (s, 2H), 4.31 (d, 1H), 4.37 (d, 1H), 7.12 (td, 1H), 7.25 (t, 1H), 7.36 (t, 1H), 7.43 (d, 1H), 7.46-7.56 (m, 2H), 7.55 (d, 1H), 8.18-8.28 (m, 1H).

### Compound 32: 8-Fluoro-3′,4′,5′,6′-tetrahydro-11H-spiro[dibenzo[b,f]thiepin-10,2′-[1,4]oxazine]

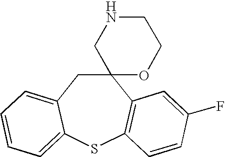

To a stirred and cooled (ice bath) suspension of LiAlH₄ powder (19 mg, 0.49 mmol, Aldrich) in anhydrous THF (2 mL) was added dropwise a solution of 8-fluoro-3′,4′-dihydro-11H-spiro[dibenzo[b,f]thiepin-10,2′-[1,4]oxazin]-5′(6′H)-one (Compound 31, 130 mg, 0.41 mmol) in anhydrous THF (1 mL). The reaction mixture was refluxed for 1 h. After cooling, water and diethyl ether were added, the two phases were separated and the organic phase was washed with water and brine. After evaporation of the organic layer, the residue was purified by silica gel chromatography (from 1/0 to 95/5 EtOAc/MeOH) to give the title compound (50 mg); MS (ESI) m/z: 302 [M+H]⁺; ¹H NMR (500 MHz, DMSO-d₆): δ 2.61 (d, 1H), 2.69 (d, 1H), 2.81-2.94 (m, 2H), 3.64 (dd, 1H), 3.83 (d, 1H), 3.88 (d, 1 H), 4.15 (td, 1H), 7.04 (td, 1H), 7.19 (t, 1H), 7.33 (t, 1H), 7.44 (dd, 1H), 7.50-7.60 (m, 3H).

### Compound 33: 1′-Methyl-5,11-dihydrospiro[dibenzo[a,d]cycloheptene-10,4′-piperidine]

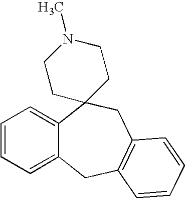

A mixture of zinc (0.121 g, 1.87 mmol), HgCl₂ (9 mg, 0.034 mmol), concentrated HCl (0.043 mL) was shaken for 5 minutes at room temperature. Then water (0.092 mL), concentrated HCl (0.216 mL) and a solution of 1′-methylspiro[dibenzo[a,d]cycloheptene-10,4′-piperidin]-11(5H)-one (Intermediate 28, 0.100 g, 0.34 mmol) in toluene (0.12 mL) were added and the reaction mixture was refluxed for 16 hours. After cooling, water was added and the mixture was washed once with DCM. The aqueous phase was basified with 32% NH₄OH solution and extracted with ethyl acetate. The organic phase was evaporated to dryness to give a brown oil. Major impurities were removed by preparative TLC (95/3/2 ethyl acetate/methanol/triethylamine) and the resulting dark oil was purified by preparative Liquid Chromatography (MDAP FractionLynx Autopurification System™) [preparative chromatographic conditions Column: Gemini C18 AXIA, 50×21 mm, 5 μm; Mobile phase: A: NH₄HCO₃ sol. 10 mM, pH10; B: CH₃CN; Gradient:40% (B) for 1 min, 40% to 55% (B) in 9 min, 55% (B) for 2 min, 55% to 100% (B) in 0.1 min, 100% (B) for 1.9 min; Flow rate: 17 ml/min; UV range: 210-350 nm; Ionization:ES⁺; Mass range: 100-900 amu]. Two fractions containing a peak with the expected m/z ratio were collected and the evaporation of the solution containing the 1^{st} peak (with the larger area) gave the title compound as a colourless oil (3.3 mg); ¹H NMR (500 MHz, DMSO-d₆): δ 1.47 (d, 2H), 2.02 (dt, 2H), 2.27 (s, 3H), 2.42 (t, 2H), 2.62 (d, 2H), 3.26 (s, 2H), 4.11 (s, 2H), 6.97-7.24 (m, 7H), 7.43 (d, 1H). LC/MS analysis, analytical chromatographic conditions (MDAP FractionLynx Autopurification System™) [Column: Gemini C18, 50×4.6 mm, 5 μm; Mobile phase: A: NH₄HCO₃ sol. 10 mM, pH 10; B: CH₃CN; Gradient: 40% (B) for 0.5 min, 40% to 55% (B) in 4.5 min, 55% to 95% (B) in 0.1 min, 95% (B) for 1.4 min; Flow rate: 2 ml/min; UV range: 210-350 nm; Ionization: ES+; Mass range: 100-900 amu]: Rt=4.55 min, m/z: 278 [M+H]⁺.

### Compound 34: 11H-spiro[dibenzo[b,f]oxepin-10,3′-pyrrolidine]

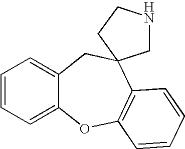

The title compound was also prepared.

### Biological Assay

### a) 5HT_{2A} Antagonist Assay

Adherent SH-SY5Y cells stably expressing the recombinant human 5-HT_{2A} were maintained in culture at 37° C. under 5% CO₂ in Alpha Minimum Essential Medium+ribonucleosides (Gibco Invitrogen,) supplemented with 10% dialysed foetal calf serum and 400 micrograms geneticin. SH-SY5Y are neuroblastoma and are commercially available from the American Type Culture Collection (ATCC), SH-SY5Y cells, expressing 5-HT_{2A} receptors, were seeded into black walled clear-base 384-well plates at a density of 16,000 cells per well and cultured overnight at 37° C. under 5% CO₂. Media was aspirated off and the cells were then incubated with HBSS medium (CaCl₂.2H₂O1.26Mm, Glucose 5.55 mM, KCl 5.36 mM MgSO₄(anhyd) 0.81 mM, NaCl 136.89 mM, KH₂PO₄(anhyd) 0.41 mM, HEPES 20 mM, NaHCO₃ 4.16 mM) containing the cytoplasmic calcium indicator, Fluo-4 in the acetylmethyl form (4 mM), 2.5 mM Probenecid and 250 uM Brilliant Black (Molecular Devices) at 37° C. for 60 min. The loaded cells were then incubated with test compound for 30 min at 37° C. The plates were then placed into a FLIPR (Molecular Devices, UK) for testing in antagonist mode, where a pre-determined concentration of 5-HT (approximately 4xEC50) was added while cell fluorescence (λex 488 nm, λem 540 nm) was monitored.

Using assay a), some of the supporting compounds gave an fpki against 5HT_{2A} of greater than 5.5.

### b) H₁ Antagonist Assay

Adherent Chinese Hamster Ovary (CHO) cells stably expressing the recombinant human H₁ receptor were maintained in culture at 37° C. under 5% CO₂ in Alpha Minimum Essential Medium without ribonucleosides (Gibco Invitrogen), supplemented with 10% dialysed foetal calf serum and 200 mM Glutamine. These cells, expressing the human H₁ receptor, were snap frozen and stored ready for assay. 24 or 72 hours prior to assay the cells were seeded into black walled clear-base 384-well plates at a density of 12,000 or 4 000 cells per well (respectively) and cultured at 37° C. under 5% CO₂. Cell seeding densities result in a confluent monolayer of cells at a time point of approximately 24 hours for 12 00 cells or 72 hours for 4 000 cells. Media was aspirated off and the cells were then incubated with HBSS medium (CaCl₂.2H₂O 1.26 Mm, Glucose 5.55 mM, KCl 5.36 mM MgSO₄(anhyd) 0.81 mM, NaCl 136.89 mM, KH₂PO₄(anhyd) 0.41 mM, HEPES 20 mM, NaHCO₃ 4.16 mM) containing the cytoplasmic calcium indicator, Fluo-4 in the acetylmethyl form (4 mM), 2.5 mM Probenecid and 250 uM Brilliant Black (Molecular Devices) at 37° C. for 60 min. The loaded cells were then incubated with test compound for 30 min at 37° C. The plates were then placed into a FLIPR (Molecular Devices, UK) for testing in antagonist mode, where a pre-determined concentration of Histamine (approximately 4xEC50) was added while cell fluorescence (λex 488 nm, λem 540 nm) was monitored.

Using assay b), all the supporting compounds gave an fpki against H₁ of greater than 5.5.

1-22. (canceled)

23. A compound of formula (I) or a pharmaceutically acceptable salt thereof: 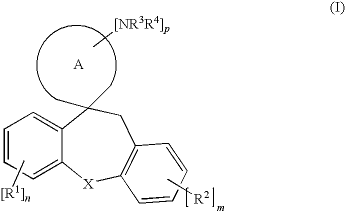
wherein:
X is CH₂, O, or S;
n is 0, 1 or 2;
m is 0, 1 or 2;
p is 0 or 1;
when present, R¹ is independently selected from the group consisting of halogen, C₁₋₃alkyl and C₁₋₃alkoxy;
when present, R² is independently selected from the group consisting of halogen, C₁₋₃alkyl and C₁₋₃alkoxy;
when p is 0, A is a spiro 5-6 membered saturated or partially unsaturated heterocyclic ring containing at least one nitrogen atom and optionally containing an additional heteroatom selected from N, S and O, the ring being optionally substituted by one or more groups independently selected from oxo and C₁₋₃alkyl;
when p is 1, A is a spiro 5-6 membered saturated or partially unsaturated carbocyclic ring;
when present, R³ and R⁴ are each independently selected from the list consisting of hydrogen and C₁₋₃alkyl; or
R³ and R⁴ together with the nitrogen to which they are attached, form a 4-6 membered saturated or partially unsaturated ring optionally containing one or more additional heteroatoms selected from O, N and S, wherein the ring is optionally substituted by one or more groups independently selected from oxo and C₁₋₃alkyl.

24. The compound according to claim 23 or a pharmaceutically acceptable salt thereof, wherein X is CH₂ or S.

25. The compound according to claim 23 or a pharmaceutically acceptable salt thereof, wherein n is 0 or 1.

26. The compound according to claim 23 or a pharmaceutically acceptable salt thereof, wherein n is 0.

27. The compound according to claim 23 or a pharmaceutically acceptable salt thereof, wherein m is 0 or 1.

28. The compound according to claim 23 or a pharmaceutically acceptable salt thereof, wherein m is 0.

29. The compound according to claim 23 or a pharmaceutically acceptable salt thereof, wherein R¹ is halogen.

30. The compound according to claim 23 or a pharmaceutically acceptable salt thereof, wherein R² is halogen.

31. The compound according to claim 23 or a pharmaceutically acceptable salt thereof, wherein when p is 1, R³ and R⁴ together with the nitrogen to which they are attached, form a 4-6 membered saturated ring optionally containing one additional heteroatom selected from O, N and S, wherein the ring is optionally substituted by one or more groups independently selected from oxo and C₁₋₃alkyl.

32. The compound according to claim 23 or a pharmaceutically acceptable salt thereof, wherein when p is 1, R³ and R⁴ together with the nitrogen to which they are attached, form a 5-6 membered saturated ring optionally containing one or more additional heteroatoms selected from O, N and S, wherein the ring is optionally substituted by one or more groups independently selected from oxo and C₁₋₃alkyl.

33. The compound according to claim 23 or a pharmaceutically acceptable salt thereof, wherein when p is 1, A is cyclopentyl.

34. A method of treatment of a disease or condition mediated by antagonism of the H₁ receptor in a human, which comprises administering to said human a therapeutically effective amount of the compound or a pharmaceutically acceptable salt thereof, as claimed in claim 23.

35. The method as claimed in claim 34, wherein the disease or condition is a sleep disorder.

36. A pharmaceutical composition comprising the compound or a pharmaceutically acceptable salt thereof, as claimed in claim 23, and a pharmaceutically acceptable carrier or excipient.

37. A process for preparing the pharmaceutical composition as defined in claim 36, the process comprising mixing the compound as claimed in claim 23 or a pharmaceutically acceptable salt thereof and a pharmaceutically acceptable carrier or excipient.
